(19) United States
(12) Patent Application Publication
(10) Pub. No.: US 2015/0011484 A1
(43) Pub. Date: Jan. 8, 2015

(54) PRODRUGS OF D-GAMMA-GLUTAMYL-D-TRYPTOPHAN AND D-GAMMA-GLUTAMYL-L-TRYPTOPHAN

(71) Applicant: APOTEX TECHNOLOGIES, INC., TORONTO (CA)

(72) Inventors: Tim Fat Tam, Vaughan (CA); Regis Leung-Toung, Mississauga (CA); Yinsheng Wang, Toronto (CA); Yanqing Zhao, Richmond Hill (CA); Tao Xin, Vaughan (CA); Wanren Li, Toronto (CA); Jolanta Maria Wodzinska, Mississauga (CA); Vrajlal S. Rabadia, Mississauga (CA); Birenkumar Shah, Brampton (CA); Christopher John Feeney, Toronto (CA)

(73) Assignee: APTOEX TECHNOLOGIES, INC., TORONTO, ON (CA)

(21) Appl. No.: 14/008,951

(22) PCT Filed: Mar. 30, 2012
(86) PCT No.: PCT/CA12/00327

§ 371 Date: Sep. 17, 2014

Related U.S. Application Data
(60) Provisional application No. 61/470,470, filed on Mar. 31, 2011.

(51) Int. Cl. C07K 5/02

(52) U.S. Cl. CPC C07K 5/0215
USPC 514/21.91; 548/507

(57) ABSTRACT

The present invention provides pro-drugs of D-gamma-glutamyl-[D/L]-tryptophan, said pro-drugs are compounds of Formula I or pharmaceutically acceptable salts thereof, wherein G is C₁-C₈ alkyl or benzyl, T is C₁-C₈ alkyl or benzyl, and * is a chiral carbon in a (R) or (S) configuration, provided that when * is in the (R) configuration, at least one of G and T is C₅-C₈ alkyl; and use of compounds of Formula I in a pharmaceutical composition.

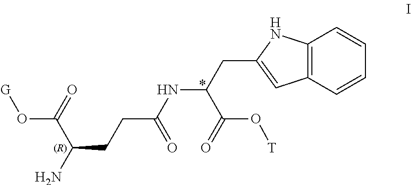

### TECHNICAL FIELD

This invention relates to the field of prodrugs of dipeptides and more particularly to the field of prodrugs of the dipeptides of D-gamma-glutamyl-D-tryptophan (H-D-Glu(D-Trp-OH)—OH) and D-gamma-glutamyl-L-tryptophan (H-D-Glu(L-Trp-OH)—OH).

### BACKGROUND

A prodrug is a compound that is modified in the body after its administration to provide an active drug. Depending on the therapeutic use and mode of administration, a prodrug may be used orally, for injection, intranasally, or in an inhaler formulation directed at lung tissues (Rautio et al. Nature Reviews Drug Discovery 7, 255-270 (February 2008). The use of prodrug compounds in an inhaler formulation directed at the lung tissue has been reviewed (Proceedings Of The American Thoracic Society Vol 1 2004, How the Lung Handles Drugs, Pharmacokinetics and Pharmacodynamics of Inhaled Corticosteroids, Julia Winkler, Guenther Hochhaus, and Hartmut Derendorf 356-363; H. Derendorf et al., Eur Respir J 2006; 28: 1042-1050).

For inhaler and intranasal means of administration, the minimization of oral bioavailability and systemic side effects by rapid clearance of absorbed active drug may be some of the design considerations. A prodrug designed for oral administration may prefer an improvement to oral bioavailability upon oral administration to animals, and appropriate chemical stability in simulated digestive fluids at pH 1.2 (also known as simulated gastric fluids) or pH 5.8 or 6.8 (also known as the simulated intestinal fluids). For prodrugs that are used in injection, the aqueous solubility of the compound is an important consideration.

The screening criteria for prodrugs depend on its mode of administration. However, a prodrug that can be readily hydrolyzed to the active drug in a human blood is a positive feature upon administration. Human blood has esterases that are capable of biotransforming some ester derivatives to the active drug (Derek Richter and Phyllis Godby Croft, Blood Esterases, Biochem J. 1942 December; 36(10-12): 746-757; Williams F M. Clinical significance of esterases in man. Clin Pharmacokinet. 1985 September-October; 10(5):392-403). In addition, prodrugs can be bioconverted in a human liver to the active drug (Baba et al., The pharmacokinetics of enalapril in patients with compensated liver cirrhosis Br J Clin Pharmacol. 1990 June; 29(6):766-9). Thus, regardless of the mode of administration, human hepatocyte and blood biotransformation results may be used to evaluate ester prodrugs.

D-Isoglutamyl-D-tryptophan or D-gamma-glutamyl-D-tryptophan (also known as H-D-Glu(D-Trp-OH)—OH or Apo805) is a synthetic hemoregulatory dipeptide developed for the treatment of autoimmune diseases including psoriasis (Sapuntsova, S. G., et al. (May 2002), Bulletin of Experimental Biology and Medicine, 133(5), 488-490). The sodium salt of H-D-Glu(D-Trp-OH)—OH (thymodepressin) is considered an effective treatment for psoriasis (U.S. Pat. No. 5,736,519), and is available as an injection ampoule in Russia.

D-Isoglutamyl-L-tryptophan or D-gamma-glutamyl-L-tryptophan (also known as H-D-Glu(L-Trp-OH)—OH or SCV-07 is reported as useful for modulating the immune system of a patient (U.S. Pat. No. 5,744,452), and useful for treating: lung cancer (WO 2009/025830A1), tuberculosis (WO 2003/013572 A1), genital viral infections (WO 2006/076169), melanoma (WO 2007/123847), hemorrhagic viral infections (WO 2006/047702), respiratory viral infections (WO 2005/112639), hepatitis C (WO 2010/017178), and injury or damage due to disease of mucosa (WO 2008/100458). SCV-07 is also reported as a vaccine enhancer (WO 2006/116053).

### SUMMARY

This invention is based, at least in part, on the discovery of prodrugs of D-gamma-glutamyl-D-tryptophan (Apo805) and D-gamma-glutamy; -L-tryptophan (SCV-07) and in particular, prodrugs that are more lipophilic than Apo805 and SCV-07. Without being bound by theory, it is believed that a prodrug which is more lipophilic than Apo805 or SCV-07 may be a prodrug that is more rapidly and more efficiently converted to Apo805 or SCV-07, respectively, in-vivo.

An example of a prodrug compound of the present invention is Apo804. Apo804 has a peptide sequence of H-D-Glu(D-Trp-OMe)-O—CH₂Ph and is a prodrug of Apo805. Apo804 is a stable chemical entity. Apo804 is more lipophilic than Apo805 and has a higher log D_{7.4}. In pharmacokinetic studies in rats, Apo804 shows improved oral bioavailability when compared with Apo805. Further evaluation in human cryopreserved hepatocyte showed that 31% of Apo805 is formed from Apo804 over a period of 4 hours.

Illustrative embodiments of the present invention provide a compound of Formula I:

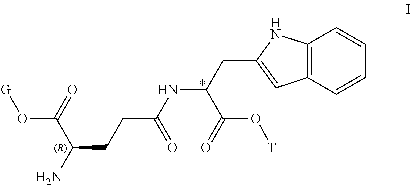

or a pharmaceutically acceptable salt thereof, wherein G is selected from the group consisting of: C₁-C₈ alkyl and benzyl; T is selected from the group consisting of: C₁-C₈ alkyl and benzyl; and * is a chiral carbon that is either in an (R) configuration or an (S) configuration, provided that when * is in the (R) configuration, at least one of G and T is C₅-C₈ alkyl.

Illustrative embodiments of the present invention provide a compound described herein wherein G is selected from the group consisting of: C₅-C₈ alkyl.

Illustrative embodiments of the present invention provide a compound described herein wherein T is selected from C₅-C₈ alkyl.

Illustrative embodiments of the present invention provide a compound described herein wherein * is in the (R) configuration.

Illustrative embodiments of the present invention provide a compound described herein wherein * is in the (S) configuration.

Illustrative embodiments of the present invention provide a compound described herein wherein G is isoamyl, T is isoamyl and * is in the (R) configuration.

Illustrative embodiments of the present invention provide a compound described herein wherein G is isoamyl, T is isoamyl and * is in the (S) configuration.

Illustrative embodiments of the present invention provide a compound described herein wherein G is heptyl, T is heptyl and * is in the (S) configuration.

Illustrative embodiments of the present invention provide a compound described herein wherein G is pentyl, T is pentyl and * is in the (S) configuration.

Illustrative embodiments of the present invention provide a compound described herein wherein G is hexyl, T is hexyl and * is in the (S) configuration.

Illustrative embodiments of the present invention provide a compound described herein wherein G is isoamyl, T is pentyl and * is in the (R) configuration.

Illustrative embodiments of the present invention provide a compound described herein wherein G is isoamyl, T is heptyl and * is in the (R) configuration.

Illustrative embodiments of the present invention provide a compound described herein wherein G is isoamyl, T is ethyl and * is in the (R) configuration.

Illustrative embodiments of the present invention provide a compound described herein wherein G is ethyl, T is ethyl and * is in the (S) configuration.

Illustrative embodiments of the present invention provide a compound described herein wherein G is ethyl, T is isoamyl and * is in the (S) configuration.

Illustrative embodiments of the present invention provide a compound described herein wherein G is ethyl, T is isoamyl and * is in the (R) configuration.

Illustrative embodiments of the present invention provide a compound described herein wherein G is benzyl, T is isoamyl and * is in the (R) configuration.

Illustrative embodiments of the present invention provide a compound described herein wherein G is benzyl, T is isoamyl and * is in the (S) configuration.

Illustrative embodiments of the present invention provide a pharmaceutical composition comprising a compound described herein and a pharmaceutically acceptable excipient.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

FIG. 1 illustrates the ACD physchem speciation calculation of the dipeptide H-D-Glu-(D-Trp-OH)—OH using estimated pKas of the acid and amine groups. The chemical structure of H₂L and H₃L are shown in the Figure. H₂L is the zwitterion species of H-D-Glu-(D-Trp-OH)—OH.

FIG. 2 illustrates the ACD physchem speciation calculation of the dipeptide H-D-Glu-(D-Trp-OMe)-OH using estimated pKas of the acid and amine groups. The chemical structure of H₂L and H₃L are shown in the Figure. H₃L is the zwitterion species of H-D-Glu-(D-Trp-OMe)-OH.

FIG. 3 illustrates the ACD physchem speciation calculation of the dipeptide H-D-Glu-(D-Trp-O-isoamyl)-O-isoamyl using estimated pKas of the acid and amine groups. The chemical structure of H₂L and H₃L are shown in the Figure. H₂L is the neutral species of H-D-Glu-(D-Trp-O-isoamyl)-O-isoamyl and H₃L is the amino salt species wherein the amino group carries a positive charge.

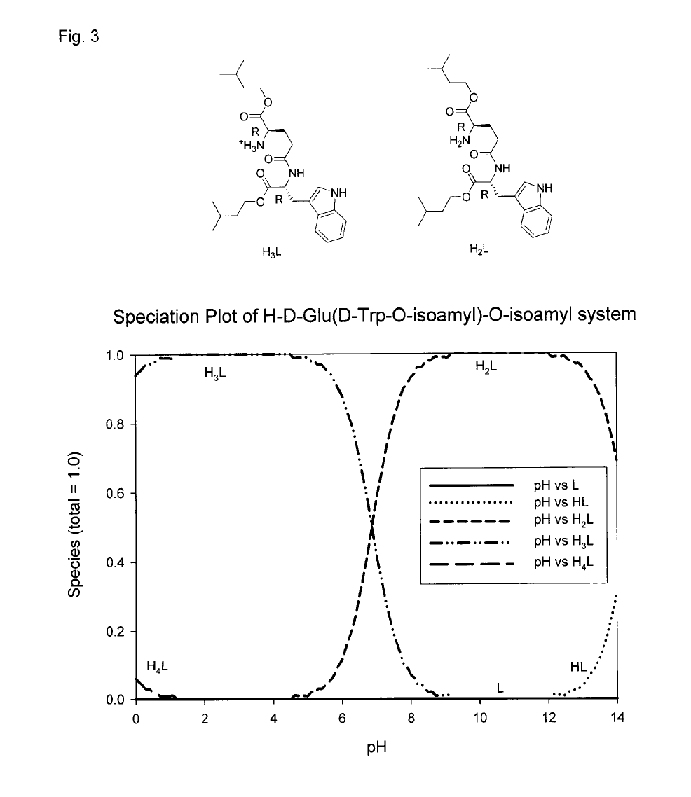

FIG. 4 shows the average (n=5) concentration of Apo805 (H-D-Glu(D-Trp-OH)—OH) in plasma after oral dosing of H-D-Glu-(D-Trp-O-isoamyl)-β-isoamyl (Apo848) and Apo805 monopotassium salt (Apo805K1) (5 mg/kg) to rats demonstrating enhanced bioavailability of the pro-drug.

### DETAILED DESCRIPTION

As used herein, the term “alkyl” means a branched or unbranched saturated hydrocarbon chain. Non-limiting, illustrative examples of alkyl moieties include, methyl, ethyl, propyl, isopropyl, n-propyl, butyl, sec-butyl, isobutyl, n-pentyl, hexyl, octyl and the like. When the terminology “Cₓ—C_{y}”, where x and y are integers, is used with respect to alkyl moieties, the ‘C’ relates to the number of carbon atoms the alkyl moiety. For example, methyl may be described as a C₁ alkyl and isobutyl may be described as a O₄ alkyl. All specific integers and ranges of integers within each range are specifically disclosed by the broad range. For example, C₁-C₈, specifically includes the following: C₁, C₂, C₃, C₄, C₅, C₆, O₇, C₈, C₁-C₂, C₁-C₃, C₁-C₄, C₁-C₅, C₁-C₆, C₁-C₇, C₁-C₈, C₂-C₃, C₂-C₄, C₂-C₅, C₂-C₆, C₂-C₇, C₂-C₈, C₃-C₄, C₃-C₅, C₃-C₆, C₃-C₇, C₃-C₈, C₄-C₅, C₄-C₆, C₄-C₇, C₄-C₈, C₅-C₆, C₅-C₇, C₅-C₈, C₆-C₇, C₆-C₈, and C₇-C₈. Another example is C₅-C₈ specifically includes C₅, C₆, C₇, C₈, C₅-C₆, C₅-C₇, C₅-C₈, C₆-C₇, C₆-C₈, and C₇-C₈.

The following acronyms and/or shorthand notation are also used herein.

| Acronym and/or Shorthand | Explanation of Acronym and/or Shorthand |
|---|---|
| EDCl | 1-ethyl-3-(3-dimethylaminopropyl) |
| | carbodiimide)hydrochloride |
| DIPEA | diisopropylethylamine |
| DMF | dimethylformamide |
| DMSO | dimethylsulfoxide |
| RT | room temperature |
| HOSu | hydroxysuccinimide |
| Boc-D-Glu(O-Bzl)-OH | 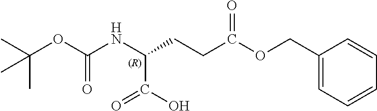 |
| Boc-D-Glu(OH)-O-isoamyl | 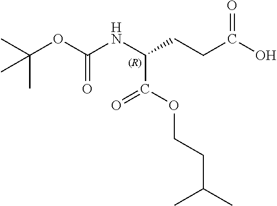 |
| Boc-D-Glu-OBzl | 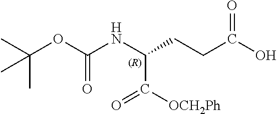 |
| Boc-D-Glu(O-Bzl)-O-isoamyl | 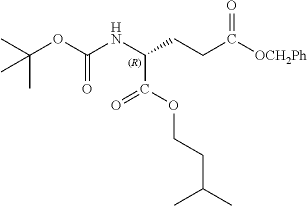 |
| H-D-Glu(D-Trp-OH)-OH | 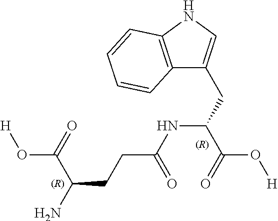 D-gamma-glutamyl-D-tryptophan |
| H-D-Glu(L-Trp-OH)-OH | 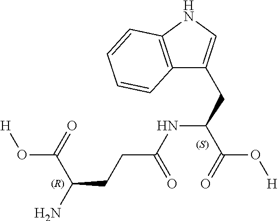 D-gamma-glutamyl-L-tryptophan |
| H-D-Glu(Trp-OH)-OH | 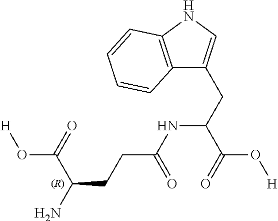 (D-gamma-glutamyl-tryptophan where the stereochemistry at the tryptophan unit is not defined) |
| H-D-Glu(D-Trp-O-heptyl)-O-isoamyl | 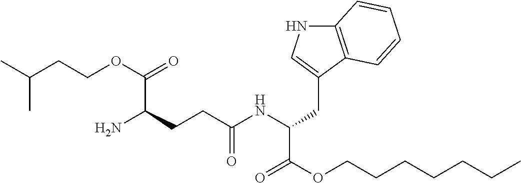 [SMILES: CCCCCCCOC(=O)[C@@H](Cc1c[nH]c2ccccc12)NC(=O)CC[C@@H](N)C(=O)OCCC(C)C] |
| H-D-Trp-O-heptyl hydrochloride | 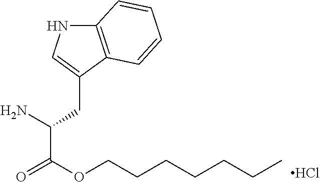 [SMILES: CCCCCCCOC(=O)[C@H](N)Cc1c[nH]c2ccccc12 ; Cl] |
| H-D-Trp-O-pentyl hydrochloride | 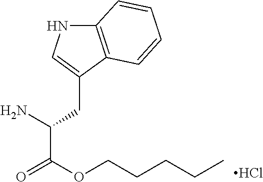 [SMILES: CCCCCOC(=O)[C@H](N)Cc1c[nH]c2ccccc12 ; Cl] |
| H-D-Glu(D-Trp-O-pentyl)-O-isoamyl hydrochloride | 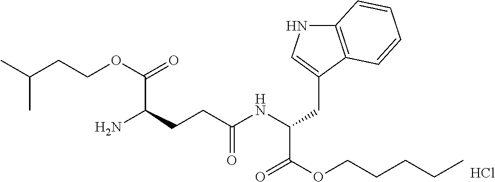 [SMILES: CCCCCOC(=O)[C@@H](Cc1c[nH]c2ccccc12)NC(=O)CC[C@@H](N)C(=O)OCCC(C)C ; Cl] |
| H-D-Glu(D-Trp-OEt)-O-isoamyl hydrochloride | 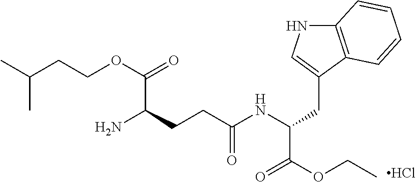 [SMILES: CCOC(=O)[C@@H](Cc1c[nH]c2ccccc12)NC(=O)CC[C@@H](N)C(=O)OCCC(C)C ; Cl] |
| Boc-D-Glu(D-Trp-O-heptyl)-O-isoamyl | 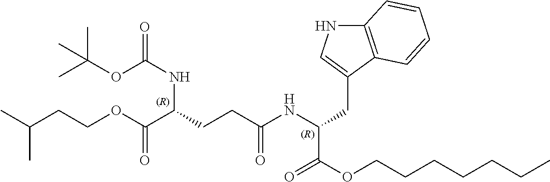 |
| Boc-D-Glu(D-Trp-O-Et)-O-isoamyl | 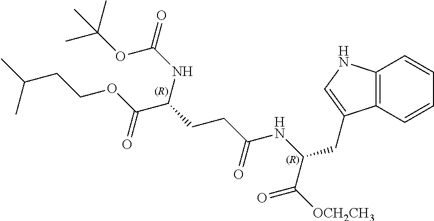 |

Compounds of the present invention may be described by Formula I:

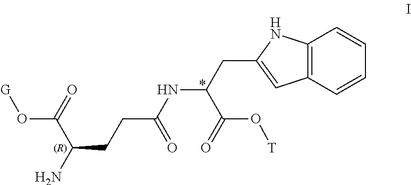

wherein G is selected from the group consisting of: C₁-C₈ alkyl and benzyl; T is selected from the group consisting of: C₁-C₈ alkyl and benzyl; and * is a chiral carbon that is either in an (R) configuration or an (S) configuration, provided that when * is in the (R) configuration, at least one of G and T is C₅-C₈ alkyl.

Compounds of Formula I include a subset termed Formula IA:

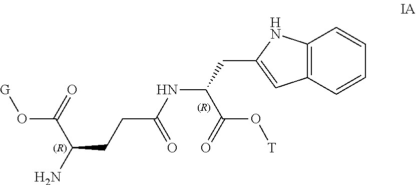

wherein * is in the (R) configuration; G is selected from the group consisting of: C₁-C₈ alkyl and benzyl; T is selected from the group consisting of: C₁-C₈ alkyl and benzyl; and at least one of G and T is C₅-C₈ alkyl.

Specific examples of Formula IA include, but are not limited to: G is ethyl and T is isoamyl; G is isoamyl and T is isoamyl; G is isoamyl and T is ethyl; G is isoamyl and T is isoamyl; G is benzyl and T is isoamyl; and G is isoamyl and T is benzyl.

Further non-limiting examples of compounds Formula IA include:

a HCl salt in which G is ethyl and T is isoamyl, termed ethyl (2R)-2-amino-5-({(2R)-3-(1H-indol-3-yl)-1-[(4-methylpentyl)oxy]-1-oxopropan-2-yl}amino)-5-oxopentanoate hydrochloride. An alternative name is the HCl salt of the peptide H-D-Glu-(D-Trp-O-isoamyl)-OEt;

a HCl salt in which G is isoamyl and T is ethyl, termed 3-methylbutyl (2R)-2-amino-5-{[(2S)-1-ethoxy-3-(1H-indol-3-yl)-1-oxopropan-2-yl]amino}-5-oxopentanoate hydrochloride. An alternative name is the HCl salt of the peptide H-D-Glu-(D-Trp-O-Et)-O-isoamyl;

an ester wherein G is isoamyl and T isoamyl, termed 3-methylbutyl (2R)-2-amino-5-{[(2R)-3-(1H-indol-3-yl)-1-(3-methylbutoxy)-1-oxopropan-2-yl]amino}-5-oxopentanoate. Alternative names include: D-gamma-glutamyl-D-tryptophan diisoamyl ester, and H-D-Glu(D-Trp-O-isoamyl)-O-isoamyl. The structure of this compound is provided below:

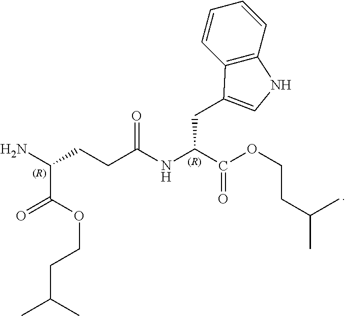

Compounds of Formula I include a subset termed Formula IB:

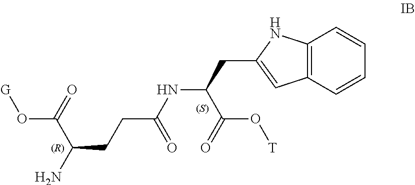

wherein * is in the (S) configuration, G is selected from the group consisting of: C₁-C₈ alkyl and benzyl; T is selected from the group consisting of: C₁-C₈ alkyl and benzyl.

Non-limiting examples of compounds of Formula IB include:

a HCl salt in which G is isoamyl and T is isoamyl, termed (2R)-5-{[(2S)-3-(1H-indol-3-yl)-1-(3-methylbutoxy)-1-oxopropan-2-yl]amino}-1-(3-methylbutoxy)-1,5-dioxopentan-2-aminium chloride. Alternative names for this salt include: D-gamma-glutamyl-L-tryptophan diisoamyl ester hydrochloride; and H-D-Glu-(L-Trp-O-isoamyl)-O-isoamyl.HCl;

a HCl salt in which G is heptyl and T is heptyl, termed heptyl (2R)-2-amino-5-{[(2S)-1-(heptyloxy)-3-(1H-indol-3-yl)-1-oxopropan-2-yl]amino}-5-oxopentanoate hydrochloride. Alternative names for this salt include: D-gamma-glutamyl-L-tryptophan di-n-heptyl ester hydrochloride; and H-D-Glu-(L-Trp-O-heptyl)-O-heptyl. HCl;

a HCl salt in which G is pentyl and T is pentyl, termed pentyl (2R)-2-amino-5-{[(2S)-3-(1H-indol-3-yl)-1-oxo-1-(pentyloxy)propan-2-yl]amino}-5-oxopentanoate hydrochloride. Alternative names for this salt include: D-gamma-glutamyl-L-tryptophan di-n-pentyl ester hydrochloride; and H-D-Glu-(L-Trp-O-pentyl)-O-pentyl.HCl;

a HCl salt in which G is hexyl and T is hexyl, termed hexyl (2R)-2-amino-5-{[(2S)-1-(hexyloxy)-3-(1H-indol-3-yl)-1-oxopropan-2-yl]amino}-5-oxopentanoate hydrochloride. Alternative names for this salt include: D-gamma-glutamyl-L-tryptophan di-n-hexyl ester hydrochloride; and H-D-Glu-(L-Trp-O-hexyl)-O-hexyl.HCl;

a HCl salt in which G is ethyl and T is isoamyl, termed ethyl (2R)-2-amino-5-({(2S)-3-(1H-indol-3-yl)-1-[(4-methylpentyl)oxy]-1-oxopropan-2-yl}amino)-5-oxopentanoate hydrochloride. An alternative name for this salt is H-D-Glu-(L-Trp-O-ethyl)-O-isoamyl.HCl.

### General Processes for Preparation of a Compound of Formula I

Compounds of Formula I wherein G and T are the same alkyl group may be prepared by the following processes (Process A and Process B).

Process A may be used for the preparation of a compound of Formula IA wherein G=T.

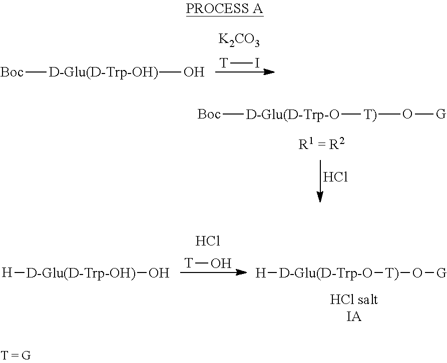

Process A is a method used to prepare a compound of formula IA wherein G and T are the same alkyl. In process A, the dipeptide Boc-D-Glu-(D-Trp-OH)—OH may be treated with potassium carbonate and T-I to give the diester Boc-D-Glu-(D-Trp-O-G)-O-T wherein G and T are the same alkyl. T-I is the reagent alkyl iodide. Deprotection of the Boc group with HCl in an inert solvent such as dioxane, or ethyl acetate affords the compound of Formula IA wherein G and T are the same. Alternatively, the compound of Formula IA wherein G and T are the same is prepared from the reaction of H-D-Glu(D-Trp-OH)—OH with the alcohol T-OH in presence of HCl. T-OH is an alkanol. In process A, the compound of formula IA is the compound of formula I with * in the (R) configuration.

An example of process A is further illustrated in example 1 below wherein T-I is 3-iodo-3-methylbutane. The reaction between Boc-D-Glu-(D-Trp-OH)—OH and T-I wherein T is 3-methylbutyl in the presence of potassium carbonate in DMF affords Boc-D-Glu-(D-Trp-O-G)-O-T wherein G=T=isoamyl. HCl deprotection of the Boc group in Boc-D-Glu-(D-Trp-O-T)-O-G in dichloromethane affords the HCl salt of formula IA wherein G=T=isoamyl. The compound of formula IA in example 1 is H-D-Glu-(D-Trp-O-isoamyl)-O-isoamyl.

Process B may be used for the preparation of a compound of Formula IB wherein G=T.

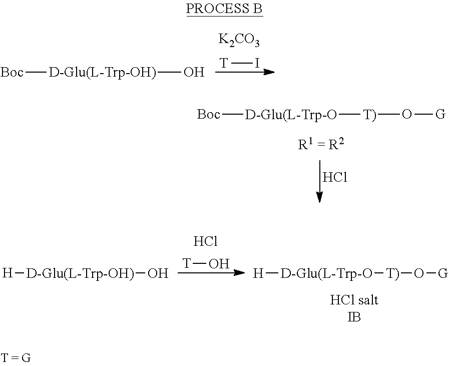

In Process B, the reaction conditions are the same as Process A with the exception that the D, L dipeptide derivative Boc-D-Glu(L-Trp-OH)—OH or H-D-Glu(L-Trp-OH)—OH is used in the preparation of a compound of Formula IB. In Process B, the compound of formula IB is a compound of formula I with * in the (S) configuration.

An example of process B is further illustrated in example 2 below. H-D-Glu(L-Trp-OH)—OH is reacted with T-OH wherein T is n-heptyl and HCl to give the HCl salt of the compound of formula IB wherein G=T=n-heptyl. The compound of formula IB in example 2 is H-D-Glu(L-Trp-O-n-heptyl)-O-n-heptyl.

Compounds of Formula I wherein T and G are independently C₁-C₈ alkyl or benzyl can be prepared by at least one of Process C and Process D.

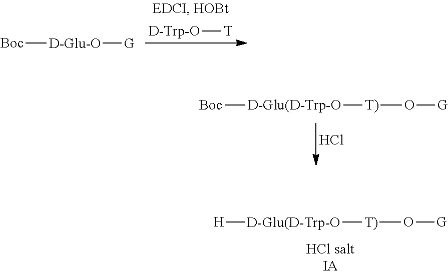

In process C, the Boc-D-Glu-O-G is coupled with D-Trp-O-T in the presence of EDCI and HOBt to give the compound Boc-D-Glu-(D-Trp-O-T)-O-G. G and T have the same definition as in the compound of formula I. HCl deprotection as described under process A affords the compound of Formula IA. In process C, the compound of formula IA is a compound of formula I with * is in the (R) configuration.

An example of process C is shown in example 6E and 6F below. Boc-D-Glu-O-G wherein G is isoamyl is coupled to D-Trp-O-T wherein T is n-heptyl with EDCI and HOBt in DMF to give the compound Boc-D-Glu-(D-Trp-O-T)-O-G wherein G is isoamyl and T is n-heptyl. HCl deprotection in an inert organic solvent such as ether affords the compound of formula IA wherein G is isoamyl and T is n-heptyl, and the compound of formula IA in example 6 is H-D-Glu-(D-Trp-O-n-heptyl)-O-isoamyl.

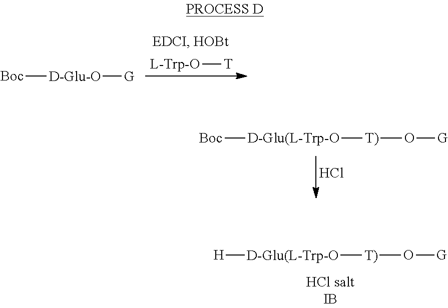

In a similar manner as Process C, Process D involves Boc-D-Glu-O-G being coupled with L-Trp-O-T to give Boc-D-Glu-(L-Trp-O-T)-O-G which is deprotected with HCl in an inert solvent to give the compound of Formula IB. In Process D, the compound of formula IB is a compound of formula I wherein * is the (S) configuration.

An example of process D is shown in example 12E and 12F below. Boc-D-Glu-O-G wherein G is ethyl is coupled to L-Trp-O-T wherein T is isoamyl with EDCI and HOBt in DMF to give the compound Boc-D-Glu-(L-Trp-O-T)-O-G wherein G is ethyl and T is isoamyl. HCl deprotection in an inert organic solvent such as ether affords the compound of formula IB wherein G is ethyl and T is isoamyl, and the compound of formula IB in example 12 is H-D-Glu-(L-Trp-O-isoamyl)-O-ethyl.

Pharmaceutically acceptable salts of compounds of the present invention include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Suitable base salts include, but are not limited to, aluminum, calcium, lithium, magnesium, potassium, sodium, zinc, and diethanolamine salts. For a review on pharmaceutically acceptable salts see Berge et al., 66 J. Pharm. Sci. 1-19 (1977).

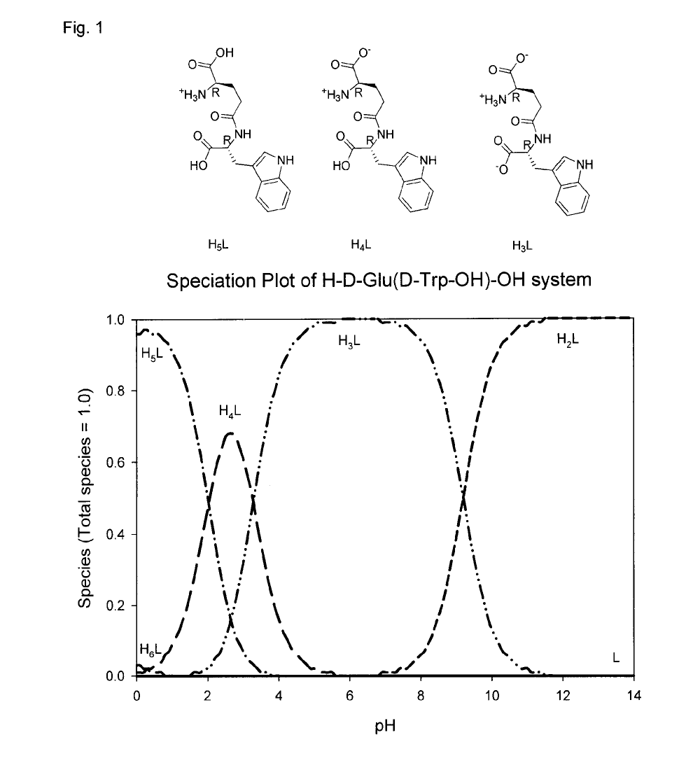

D-gamma-Glutamyl-D-tryptophan has two carboxylic acids and one amino group in the chemical structure. The speciation plot representing charged and/or neutral species against a pH scale can be computed using ACD physchem software (Advanced Chemistry Development, Inc., Toronto, Ontario, Canada). As shown in FIG. 1, the main species at pH 5.8 to 7.4 is H₃L, and thus the dipeptide D-gamma-glutamyl-D-tryptophan exists as a negatively charged carboxylate salt.

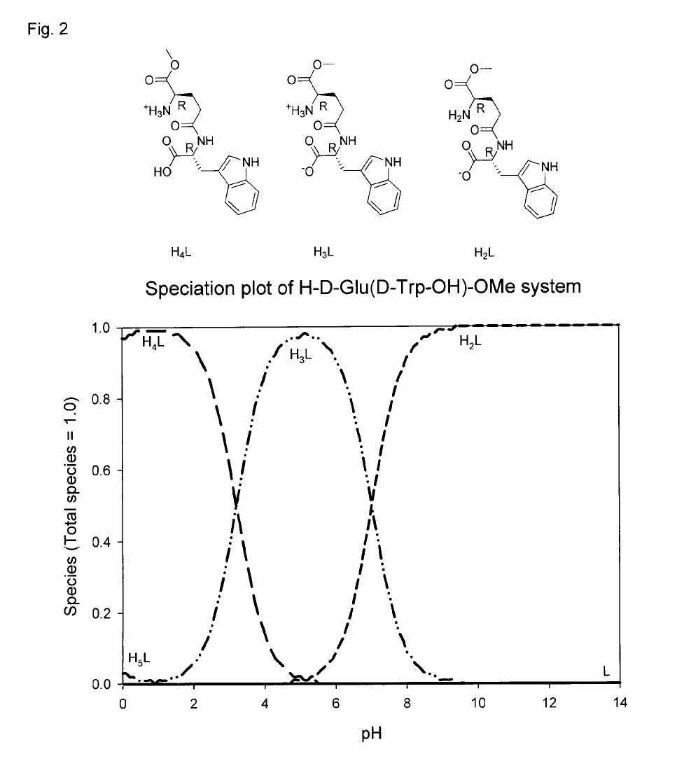

The speciation plot of the mono alkyl ester of D-gamma-glutamyl-D-tryptophan H-D-Glu(D-Trp-OMe)-OH is shown in FIG. 2. The percentage of the electrically neutral H₃L zwitterion species is pH dependent, and more of negatively charged H₂L species (one negative charge) is present at pH 7.4. For example, the computed speciation distribution of H-D-Glu(D-Trp-OMe)-OH at key pHs are shown in the Table below:

| pH | H₂L (1 −VE charge) 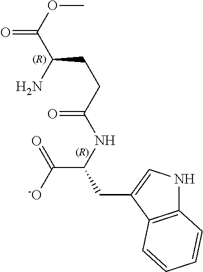 | H₃L (zwitterions) 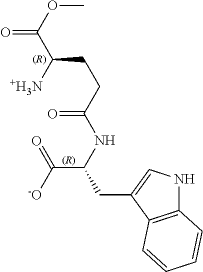 |
|---|---|---|
| 6.0 | 0.09 | 0.91 |
| 6.8 | 0.38 | 0.62 |
| 7.2 | 0.60 | 0.40 |
| 7.4 | 0.71 | 0.29 |

*Total species = 1.0 (ACD physchem v11.03). As an illustrative example, 0.09 and 0.91 in the above table means 9% and 91% of H₂L and H₃L species, respectively, present in solution at pH 6.0.

In the case of the monoalkyl ester H-D-Glu(D-Trp-OMe)-OH, the available species for intestinal absorption is a mixture of negatively charged H₂L and electrically neutral zwitterionic H₃L species at the pH range of 6.0 to 7.4.

When the prodrug is a D-gamma-glutamyl-D-tryptophan dialkyl ester such as H-D-Glu(D-Trp-O-isoamyl)-O-isoamyl, the neutral species is H₂L. The speciation at key pHs are

| pH | H₂L (neutral) 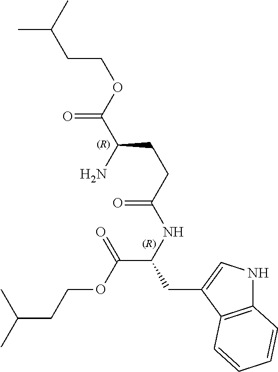 | H₃L (1 +VE charge) 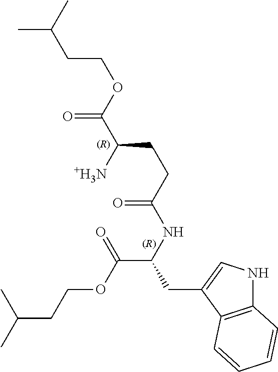 |
|---|---|---|
| 6.0 | 0.12 | 0.88 |
| 6.8 | 0.46 | 0.54 |
| 7.2 | 0.68 | 0.32 |
| 7.4 | 0.77 | 0.23 |

* Total species = 1.0 (ACD physchem v11.03). As an illustrative example, 0.12 and 0.88 in the above table means 12% and 88% of H₂L and H₃L species respectively, present in solution at pH 6.0.

Between pH 6 and 7.4, H-D-Glu(D-Trp-O-isoamyl)-O-isoamyl is a mixture of H₂L and H₃L, with H₂L being the neutral species.

D-gamma-Glutamyl-D-tryptophan dialkyl ester, in particular those with at least one C₅-C₈ alkyl ester, show improved in lipophilicity when compared to D-gamma-glutamyl-D-tryptophan C₁-C₄ dialkyl ester. A comparison of experimental log D at pH 7.4 is shown below:

| Compound | Classification | Log D_{7.4} |
|---|---|---|
| H-D-Glu(D-Trp-O-isoamyl)-O- | C₅-C₈ dialkyl | 2.1 |
| isoamyl | ester | |
| H-D-Glu(D-Trp-O—Me)—O—Me | C₁-C₄ dialkyl | 0.57 |
| | ester | |
| H-D-Glu(D-Trp-O—Me)—OH | C₁ dialkyl ester | −0.89 |
| H-D-Glu(D-Trp-OH)—OH | parent drug | −3.22 |

The use of a diisoamyl ester may improve the log D value of H-D-Glu(D-Trp-OH)—OH by more than 10⁵ fold. A prodrug may be biotransformed at multiple sites in the body to the parent drug. Examples of such sites in the body include the intestinal compartment, the blood and the liver. For a dialkyl ester prodrug, one of the possible sites of biotransformation is the liver. A more lipophilic compound may facilitate the compound reaching the human hepatocytes for biotransformation into the parent drug H-D-Glu(D-Trp-OH)—OH after intestinal absorption. As noted above, the compound H-D-Glu(D-Trp-O-isoamyl)-O-isoamyl is more lipophilic than the dimethyl ester H-D-Glu(D-Trp-O-Me)-O-Me or the monomethyl ester H-D-Glu(D-Trp-O-Me)-OH.

When H-D-Glu(D-Trp-OH)—OH diisoamyl ester and dimethyl ester are tested in human hepatocytes, the biological evaluation data supports that there is a higher percent of H-D-Glu(D-Trp-OH)—OH formed in human hepatocyte formed over a period of four hours.

TABLE 1
In vitro bioconversion of diester pro-drugs in human hepatocytes.

| | | Bioconversion |
|---|---|---|
| Compound | | to Apo805 |
| ID | Peptide sequence | in human hepatocytes |
| Apo840 | H-D-Glu(D-Trp-O—Me)—O—Me | 30% in 3 h |
| Apo848 | H-D-Glu(D-Trp-O-isoamyl)-O- | 45% in 3 h |
| | isoamyl | |

Applying the same screening technology with human hepatocytes, 50% of enalapril is biotransformed to enalaprilate in 2.9 hours. The biotransformation of enalapril to enalaprilate in liver of human patients has been reported in Br. J. Clin. Pharmacol. (1990), 29, 766-769. Hence, it can be seen that Apo848 has a similar profile of biotransformation to H-D-Glu(D-Trp-OH)—OH in human hepatocytes within 3 h as enalapril to enalaprilate.

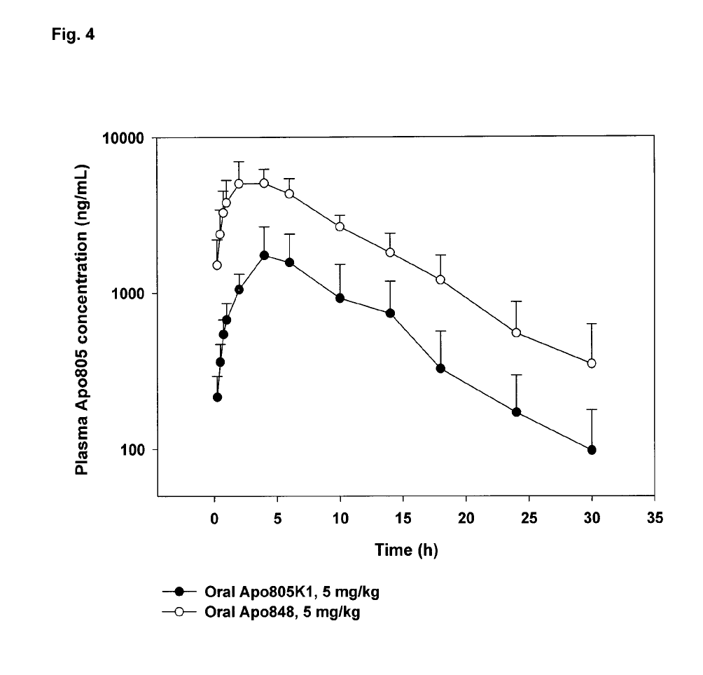

When Apo848 is tested in pharmacokinetic studies in rats, it showed improved oral exposure when compared with H-D-Glu(D-Trp-OH)—OH and the results of this study are depicted in FIG. 4 and in Example 9 below.

Compounds of the present invention or salts thereof may be formulated into a pharmaceutical formulation. Many compounds of this invention are generally water soluble and may be formed as salts. In such cases, pharmaceutical compositions in accordance with this invention may comprise a salt of such a compound, preferably a physiologically acceptable salt, which are known in the art. Pharmaceutical preparations will typically comprise one or more carriers acceptable for the mode of administration of the preparation, be it by injection, inhalation, topical administration, lavage, or other modes suitable for the selected treatment. Suitable carriers are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Compounds or pharmaceutical compositions in accordance with this invention or for use in this invention may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition according to the invention includes a therapeutically effective amount or a prophylactically effective amount. A “therapeutically effective amount” refers to an amount effective, at dosages and for periods of time necessary, to achieve the desired therapeutic result, such as improved PASI score or other suitable clinical indication known to a person of skill in the art. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as a desirable PASI score (Psoriasis Area and Severity Index) or other suitable clinical indication known to a person of skill in the art. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD₅₀ (the dose lethal to 50% of the population) and the LD₁₀₀ (the dose lethal to 100% of the population). In some circumstances however, such as in severe disease conditions, it may be necessary to administer substantial excesses of the compositions.

As used herein, a “subject” may be a human, non-human primate, rat, mouse, cow, horse, pig, sheep, goat, dog, cat, etc. The subject may be suspected of having or at risk for having psoriasis and/or atopic dermatitis and/or a medical condition wherein an agent is used in modulating the immune system. Diagnostic methods for psoriasis, atopic dermatitis and various disorders for which immune modulating compounds are used and the clinical delineation of those conditions' diagnoses are known to those of ordinary skill in the art.

### EXAMPLES

The following examples are illustrative of some of the embodiments of the invention described herein. These examples do not limit the spirit or scope of the invention in any way.

### Example 1

### Preparation of 3-methylbutyl (2R)-2-amino-5-{[(2R)-3-(1H-indol-3-yl)-1-(3-methylbutoxy)-1-oxopropan-2-yl]amino}-5-oxopentanoate hydrochloride (Apo848.HCl), H-D-Glu(D-Trp-O-isoamyl)-O-isoamyl.HCl

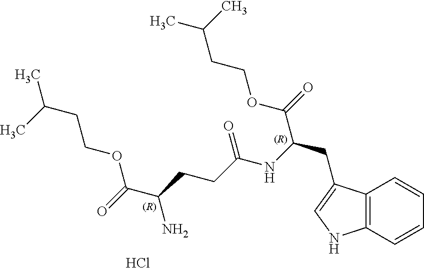

### Step 1: Preparation of Boc-D-Glu(D-Trp-O-isoamyl)-O-isoamyl

To a solution of N-(tert-butoxycarbonyl)-D-gamma-glutamyl-D-tryptophan (Boc-D-Glu(D-Trp-OH)—OH, Apo806, 4.00 g, 9.23 mmol) in DMF (30 mL) cooled in an ice-water bath was successively added anhydrous potassium carbonate (5.10 g, 36.9 mmol) and a solution of 1-iodo-3-methylbutane (4.90 mL, 36.9 mmol) in DMF (10 mL) dropwise over 10 min. The mixture was allowed to warm to RT and stirred for 18 h. The reaction mixture was poured into de-ionized water (150 mL), stirred for 30 min as a solid precipitated out. Hexanes (150 mL) was added, and the mixture was stirred for 10 min. Hexanes and water were removed via decantation, and fresh de-ionized water (100 mL) and hexanes (150 mL) were added. The mixture was stirred for an additional 15 min. The solid was collected by suction filtration, washed with hexanes (25 mL×5) and dried in a vacuum oven to afford 3-methylbutyl (2R)-2-[(tert-butoxycarbonyl)amino]-5-{[(2R)-3-(1H-indol-3-yl)-1-(3-methylbutoxy)-1-oxopropan-2-yl]amino}-5-oxopentanoate (Boc-D-Glu(D-Trp-O-isoamyl)-O-isoamyl) as a brown solid (4.50 g). Yield=85.1%; ¹H NMR (DMSO-D₆, 90 MHz) δ ppm: 10.84 (s, 1H), 8.28 (s, 1H), 6.94-7.54 (m, 6H), 3.73-4.64 (m, 6H), 3.10 (s, 2H), 1.97-2.38 (m, 2H), 1.23-1.45 (m, 17H), 0.65-0.97 (m, 12H); MS-ESI (m/z): 575 [M+1]⁺.

### Step 2: Preparation of H-D-Glu(D-Trp-O-isoamyl)-O-isoamyl.HCl

3-Methylbutyl (2R)-2-[(tert-butoxycarbonyl)amino]-5-{[(2R)-3-(1H-indol-3-yl)-1-(3-methylbutoxy)-1-oxopropan-2-yl]amino}-5-oxopentanoate (Boc-D-Glu(D-Trp-O-isoamyl)-O-isoamyl) (1.10 g, 1.92 mmol) was dissolved in dichloromethane (100 mL) and the solution was cooled in an ice-water bath. HCl gas was bubbled into the cold solution for 2 h. The reaction mixture was then allowed to warm to RT and nitrogen gas was bubbled for 30 min. Volatile materials were removed via rotary evaporation under reduced pressure. The residual solid was then dried in a vacuum oven to afford the title compound (0.67 g). Yield=69.5%. ¹H NMR (DMSO-D₆, 400 MHz) δ ppm: 10.92 (br. s, 1H), 8.47-8.62 (m, 4H), 7.48 (d, J=8.1 Hz, 1H), 7.34 (d, J=8.1 Hz, 1H), 7.17 (s, 1H), 7.07 (t, J=7.1 Hz, 1H), 6.96-7.03 (m, 1H), 4.45-4.52 (m, 1H), 4.12-4.22 (m, 2H), 3.94-4.04 (m, 3H), 3.01-3.17 (m, 2H), 2.24-2.40 (m, 2H), 1.97 (d, J=6.1 Hz, 2H), 1.61-1.71 (m, 1H), 1.42-1.55 (m, 3H), 1.28-1.39 (m, 2H), 0.89 (d, J=7.1 Hz, 6H), 0.77-0.85 (m, 6H); MS-ESI (m/z): 475 [M+1]⁺.

### Example 2

### Preparation of gamma-D-glutamyl-L-tryptophan diheptyl ester hydrochloride or heptyl (2R)-2-amino-5-{[(2S)-1-(heptyloxy)-3-(1H-indol-3-yl)-1-oxopropan-2-yl]amino}-5-oxopentanoate hydrochloride (Apo874 hydrochloride) H-D-Glu(L-Trp-O-heptyl)-O-heptyl.HCl

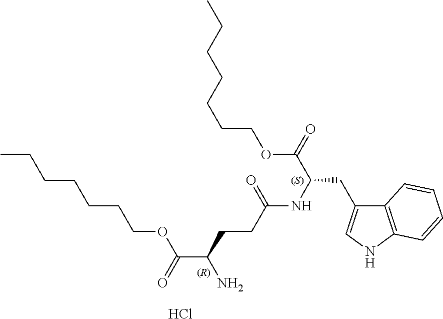

To an ice-cooled suspension of D-gamma-glutamyl-L-tryptophan (4.0 g, 12 mmol) in CH₂Cl₂ (60 mL) and heptanol (7.0 g, 60 mmol) was bubbled HCl gas. The progress of the reaction was monitored by HPLC: HPLC Column: XTerra MS, C18, 5 μm, 4.6×250 mm; Mobile phase: A=the aqueous phase: 4 mM Tris, 2 mM EDTA, pH 7.4; B=the organic phase: CH₃CN; Method gradient: Time in min-B %:0-5%, 15-90%, 25-90%;Flow rate=1 mL/min; injection volume=5 μL; λ: 222, 254, 280, 450 nm; Retention Time (RT) of starting material=5.6 min; RT of Apo874=18.6 min. After 2 h at ice-cold temperature, analysis of the reaction mixture by HPLC (area under curve, AUC) indicated presence of about 31% of the starting material. The reaction mixture was allowed to warm to ambient temperature and stirred for overnight. The reaction mixture was again cooled in ice, and 1-heptanol (7.0 g, 60 mmol) was added. HCl gas was then bubbled into the mixture and the resulting mixture was stirred for another 6 h. Nitrogen gas was bubbled into the reaction mixture, and the mixture was then evaporated to dryness in vacuo to give the title compound. A sample of Apo874 hydrochloride (1.3 g) was isolated after purification by flash column chromatography on silica gel using a solvent gradient consisting of a mixture of isopropanol and dichloromethane (7 to 100%); HPLC (AUC) purity at 280 nm=98.4%; ¹H NMR (DMSO-D₆) δ ppm: 10.92 (s, 1H), 8.55 (d, J=7.4 Hz, 1H), 8.20-8.50 (br., 3H), 7.47 (d, J=7.8 Hz, 1H), 7.34 (d, J=8.0 Hz, 1H), 7.17 (s, 1H), 7.06 (t, J=7.4 Hz, 1H), 6.98 (t, J=7.4 Hz, 1H), 4.46-4.47 (m, 1H), 4.11-4.14 (m, 2H), 3.92-3.99 (m, 3H), 3.01-3.15 (m, 2H), 2.30-2.40 (m, 1H), 2.20-2.30 (m, 1H), 1.90-2.10 (m, 2H), 1.50-1.70 (m, 2H), 1.40-1.50 (m, 2H), 1.10-1.40 (m, 16H), 0.80-0.90 (m, 6H); MS-ESI (m/z): 530 [M-HCl+1]+(free base).

### Example 3

### Preparation of 3-methylbutyl (2R)-2-amino-5-{[(2S)-3-(1H-indol-3-yl)-1-(3-methylbutoxy)-1-oxopropan-2-yl]amino}-5-oxopentanoate hydrochloride, Apo871.HCl, H-D-Glu(L-Trp-O-isoamyl)-O-isoamyl.HCl

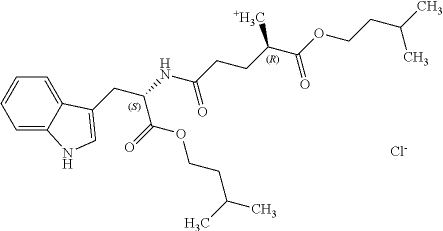

In a similar manner as described in Example 2,3-methylbutyl (2R)-2-amino-5-{[(2S)-3-(1H-indol-3-yl)-1-(3-methylbutoxy)-1-oxopropan-2-yl]amino}-5-oxopentanoate hydrochloride, Apo871 hydrochloride salt, was prepared by bubbling HCl gas into a mixture of H-D-Glu(L-Trp-OH)—OH in isoamyl alcohol. A sample was purified by flash column chromatography on silica gel using a solvent gradient consisting of a mixture of isopropanol and dichloromethane (10 to 100%). The HPLC method described in Example 2 was used. HPLC (AUC) purity at 280 nm=99.2%; ¹H NMR (DMSO-D₆) δ ppm: 10.91 (s, 1H), 8.50 (d, J=7.3 Hz, 1H), 7.2-8.2 (br., 3H), 7.47 (d, J=7.8 Hz, 1H), 7.34 (d, J=8.0 Hz, 1H), 7.17 (s, 1H), 7.07 (t, J=7.4 Hz, 1H), 6.98 (t, J=7.4 Hz, 1H), 4.45-4.47 (m, 1H), 4.13-4.17 (m, 2H), 3.96-3.99 (m, 2H), 3.83-3.86 (m, 1H), 3.01-3.15 (m, 2H), 2.33-2.35 (m, 1H), 2.23-2.25 (m, 1H), 1.87-1.94 (m, 2H), 1.64-1.67 (m, 1H), 1.46-1.52 (m, 3H), 1.29-1.34 (m, 2H), 0.87-0.89 (m, 6H), 0.79-0.82 (m, 6H); MS-ESI (m/z): 474 [M-HCl+1]+(free base).

### Example 4

### Preparation of gamma-D-glutamyl-L-tryptophan dipentyl ester hydrochloride or pentyl (2R)-2-amino-5-{[(2S)-3-(1H-indol-3-yl)-1-oxo-1-(pentyloxy)propan-2-yl]amino}-5-oxopentanoate, Apo876 hydrochloride salt or H-D-Glu(L-Trp-O-pentyl)-O-pentyl.HCl

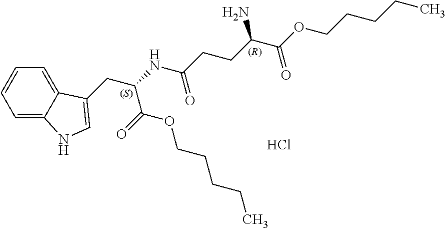

In a similar manner as described in Example 2, H-D-Glu(L-Trp-OH)—OH was reacted with HCl in n-pentanol to give pentyl (2R)-2-amino-5-{[(2S)-3-(1H-indol-3-yl)-1-oxo-1-(pentyloxy)propan-2-yl]amino}-5-oxopentanoate, Apo876 hydrochloride salt. HPLC (AUC) purity at 280 nm=99.2%; ¹H NMR (DMSO-D₆) δ ppm: 10.85 (s, 1H), 8.29 (d, J=7.4 Hz, 1H), 7.48 (d, J=7.8 Hz, 1H), 7.33 (d, J=8.0 Hz, 1H), 7.14 (s, 1H), 7.06 (t, J=7.4 Hz, 1H), 6.98 (t, J=7.4 Hz, 1H), 4.43-4.49 (m, 1H), 3.99-4.06 (m, 2H), 3.92-3.95 (m, 2H), 3.24-3.28 (m, 1H), 2.99-3.14 (m, 2H), 2.14-2.24 (m, 2H), 1.75-1.83 (m, 2H), 1.53-1.58 (m, 3H), 1.41-1.44 (m, 2H), 1.26-1.30 (m, 3H), 1.06-1.25 (m, 4H), 0.81-0.88 (m, 6H); MS-ESI (m/z): 474 [M-HCl+1]+(free base).

### Example 5

### Preparation of gamma-D-glutamyl-L-tryptophan dihexyl ester hydrochloride or hexyl (2R)-2-amino-5-{[(2S)-1-(hexyloxy)-3-(1H-indol-3-yl)-1-oxopropan-2-yl]amino}-5-oxopentanoate hydrochloride or H-D-Glu(L-Trp-O-hexyl)-O-hexyl.HCl (Apo881 hydrochloride salt)

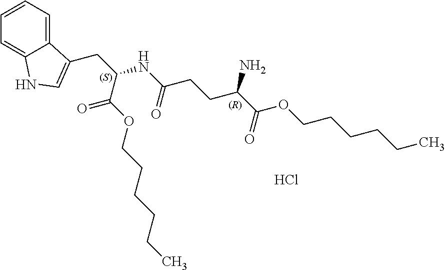

In a similar manner as described in Example 2, H-D-Glu(L-Trp-OH)—OH was reacted with HCl in hexanol to give hexyl (2R)-2-amino-5-{[(2S)-1-(hexyloxy)-3-(1H-indol-3-yl)-1-oxopropan-2-yl]amino}-5-oxopentanoate hydrochloride, Apo881 hydrochloride salt or gamma-D-glutamyl-L-tryptophan dihexyl ester hydrochloride. HPLC (AUC) purity at 280 nm=95.0%; ¹H NMR (DMSO-D₆) δ ppm: 10.91 (s, 1H), 8.46 (d, J=7.3 Hz, 1H), 6.80-7.80 (br., 3H), 7.48 (d, J=7.8 Hz, 1H), 7.34 (d, J=8.0 Hz, 1H), 7.16 (s, 1H), 7.06 (t, J=7.4 Hz, 1H), 6.98 (t, J=7.4 Hz, 1H), 4.43-4.49 (m, 1H), 4.07-4.11 (m, 2H), 3.93-3.96 (m, 2H), 3.72-3.73 (m, 1H), 3.03-3.14 (m, 2H), 2.30-2.40 (m, 1H), 2.20-2.30 (m, 1H), 1.90-2.00 (m, 1H), 1.80-1.90 (m, 1H), 1.50-1.60 (m, 2H), 1.40-1.50 (m, 2H), 1.10-1.40 (m, 12H), 0.70-0.90 (m, 6H); MS-ESI (m/z): 502 [M-HCl+1]+(free base).

### Example 6

### Preparation of H-D-Glu(D-Trp-O-heptyl)-O-isoamyl hydrochloride (Apo922.HCl)

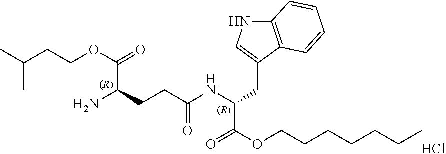

### A. Preparation of Boc-D-Trp-O-heptyl

Boc-D-Trp-OH (10.0 g, 32.8 mmol), heptanol (3.82 g, 32.8 mmol), EDCI (6.93 g, 36.1 mmol), HOBt hydrate (5.53 g, 36.1 mmol) and DIPEA (4.24 g, 32.8 mmol) were mixed in dichloromethane (100 mL) and DMF (100 mL). The reaction mixture was stirred at room temperature for overnight and then concentrated by rotary evaporation to remove dichloromethane. The residue was taken up in ethyl acetate, then successively washed with water, a saturated sodium bicarbonate solution, water, a 1N HCl solution, water and brine, then dried over magnesium sulphate. After filtration, the organic solution was concentrated to dryness and the residue was triturated with hexanes to give Boc-D-Trp-O-heptyl (7.89 g) as a white solid. Yield=60%; ¹H NMR (CDCl₃, 400 MHz) δ (ppm): 8.05 (br. s, 1H), 7.57 (d, J=8.1 Hz, 1H), 7.35 (d, J=8.1 Hz, 1H), 7.19 (t, J=7.6 Hz, 1H), 7.07-7.15 (m, 1H), 7.02 (s, 1H), 5.07 (d, J=8.1 Hz, 1H), 4.56-4.69 (m, 1H), 3.95-4.12 (m, 2H), 3.29 (br. s, 2H), 1.48-1.63 (m, 5H), 1.15-1.46 (m, 14H), 0.88 (t, J=7.1 Hz, 3H); MS-ESI (m/z): 403 [M+1]⁺.

### B. Preparation of H-D-Trp-O-heptyl hydrochloride

To a solution of Boc-D-Trp-O-heptyl (7.40 g, 18.4 mmol) in ethyl acetate (75 mL) and ether (75 mL) under ice-water bath cooling, was slowly bubbled HCl gas with stirring for 2 h until no more starting material remained as monitored by TLC. The reaction mixture was concentrated in vacuo, and then mixed with water (10 mL) and acetonitrile. The mixture was concentrated again, and the residue was triturated with ether to give H-D-Trp-O-heptyl hydrochloride (5.43 g) as an off-white solid. Yield=87%. ¹H NMR (DMSO-D₆, 400 MHz) δ (ppm): 11.10 (br. s, 1H), 8.58 (br. s, 3H), 7.51 (d, J=8.1 Hz, 1H), 7.37 (d, J=7.1 Hz, 1H), 7.24 (s, 1H), 7.10 (t, J=7.6 Hz, 1H), 6.95-7.06 (m, 1H), 4.21 (t, J=6.1 Hz, 1H), 3.88-4.10 (m, 2H), 3.15-3.37 (m, 2H), 1.35-1.50 (m, 2H), 1.03-1.31 (m, 8H), 0.86 (m, 3H); MS-ESI (m/z): 303 [M+1]⁺ (free base).

### C. Preparation of Boc-D-Glu(OBzl)-O-isoamyl

To a suspension of Boc-D-Glu(O-Bzl)-OH (5.48 g, 16.2 mmol), potassium carbonate (4.48 g, 32.5 mmol) and DMF (30 mL) at room temperature was added 1-iodo-3-methylbutane (6.43 g, 32.5 mmol). After the reaction mixture was stirred at room temperature for overnight, the solid was filtered off and washed with ethyl acetate. The filtrate was concentrated by rotary evaporation and the residue was mixed with water. The resulting solid was taken up in hexanes, and the organic solution was washed with water (2×), dried over magnesium sulphate, then filtered. The filtrate was concentrated by rotary evaporation to give Boc-D-Glu(O-Bzl)-O-isoamyl as a white solid (6.64 g) in quantitative yield. ¹H NMR (CDCl₃, 90 MHz) δ ppm: 7.03-7.56 (m, 5H), 5.12 (s, 3H), 3.87-4.50 (m, 3H), 2.25-2.63 (m, 2H), 1.83-2.20 (m, 2H), 1.23-1.75 (m, 12H), 0.91 (d, J=5.85 Hz, 6H).

### D. Preparation of Boc-D-Glu(OH)—O-isoamyl

Boc-D-Glu(O-Bzl)-O-isoamyl (6.20 g, 15.2 mmol) from above and 10% Pd/C (wet, 0.62 g) were mixed in ethyl acetate (80 mL). The reaction mixture was hydrogenated under a hydrogen gas atmosphere using a Parr apparatus at 40 psi hydrogen pressure for 4.5 h. The mixture was filtered through Celite™ and the cake was thoroughly washed with ethyl acetate. The filtrate was concentrated by rotary evaporation to give the title compound Boc-D-Glu(OH)—O-isoamyl as a sticky clear oil in quantitative yield (5.50 g). ¹H NMR (CDCl₃, 400 MHz) 8 ppm: 5.18 (d, J=7.1 Hz, 1H), 4.35 (br. s, 1H), 4.18 (t, J=7.1 Hz, 2H), 2.38-2.54 (m, 2H), 2.12-2.27 (m, 1H), 1.84-2.04 (m, 1H), 1.63-1.81 (m, 1H), 1.50-1.63 (m, 2H), 1.45 (s, 9H), 0.93 (d, J=6.1 Hz, 6H).

### E. Preparation of Boc-D-Glu(D-Trp-O-heptyl)-O-isoamyl

To a solution of Boc-D-Glu(OH)—O-isoamyl (952 mg, 3.0 mmol), H-D-Trp-O-heptyl hydrochloride (1.02 g, 3.0 mmol), EDCI (933 mg, 3.3 mmol), HOBt hydrate (505 mg, 3.3 mmol) in DMF (10 mL) under ice-water bath cooling was added DIPEA (426 mg, 3.3 mmol.). The reaction mixture was stirred at RT for overnight. The reaction mixture was diluted with ethyl acetate, and the organic phase was successively washed with water, a 1N HCl solution, water, a saturated sodium bicarbonate solution, water and brine. The organic layer was concentrated with silica gel by rotary evaporation and the residue was purified by column chromatography on silica gel with a mixture of ethyl acetate (20 to 30%) in hexanes to give Boc-D-Glu(D-Trp-O-heptyl)-O-isoamyl (1.60 g) as a pale-yellow sticky oil. Yield=83%; ¹H NMR (CDCl₃, 400 MHz) δ (ppm): 8.15 (br. s, 1H), 7.53 (d, J=6.1 Hz, 1H), 7.35 (d, J=6.1 Hz, 1H), 7.15-7.23 (m, 1H), 7.06-7.15 (m, 1H), 7.03 (br. s, 1H), 6.24 (d, J=5.1 Hz, 1H), 5.23 (d, J=6.1 Hz, 1H), 4.93 (d, J=5.1 Hz, 1H), 3.91-4.33 (m, 5H), 3.20-3.47 (m, 2H), 2.08-2.32 (m, 3H), 1.90 (d, J=7.1 Hz, 1H), 1.36-1.72 (m, 14H), 1.26 (br. s, 8H),), 0.79-1.02 (m, 9H); MS-ESI (m/z): 602 [M+1]⁺.

### F. Preparation of H-D-Glu(D-Trp-O-heptyl)-O-isoamyl hydrochloride

Boc-D-Glu(D-Trp-O-heptyl)-O-isoamyl (1.56 g, 2.6 mmol) was mixed with a 2M HCl in ether solution (15 mL) at RT and stirred for overnight. The reaction mixture was concentrated under reduced pressure by rotary evaporation. The residue was partitioned between a saturated sodium bicarbonate solution and ethyl acetate. The organic layer was dried over MgSO₄, filtered and concentrated to dryness by rotary evaporation to give a sticky oil. The oil was taken up in ether and acidified with a 2M HCl in ether solution (1.5 mL). The resulting suspension was concentrated again by rotary evaporation to give H-D-Glu(D-Trp-O-heptyl)-O-isoamyl hydrochloride (750 mg) as an off-white foam. Yield=46%; ¹H NMR (DMSO-D₆, 400 MHz) δ (ppm): 11.01 (br. s, 1H), 8.75 (br. s, 3H), 8.56 (d, J=6.1 Hz, 1H), 7.47 (d, J=7.1 Hz, 1H), 7.34 (d, J=8.1 Hz, 1H), 7.20 (br. s, 1H), 7.05 (t, J=7.6 Hz, 1H), 6.90-7.00 (m, 1H), 4.38-4.56 (m, 1H), 4.13 (t, J=6.1 Hz, 2H), 3.79-4.03 (m, 3H), 2.94-3.25 (m, 2H), 2.18-2.46 (m, 2H), 1.88-2.12 (m, 2H), 1.64 (dt, J=12.4, 6.4 Hz, 1H), 1.35-1.54 (m, 4H), 1.05-1.30 (m, 8H), 0.70-0.95 (m, 9H); MS-ESI (m/z): 502 [M+1]⁺ free base.

### Example 7

### Preparation of H-D-Glu(D-Trp-O-pentyl)-O-isoamyl hydrochloride (Apo921.HCl)

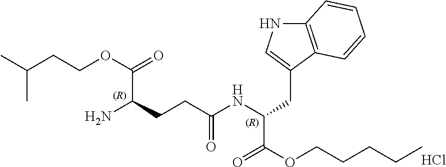

### A. Preparation of Boc-D-Trp-O-pentyl

Proceeding in a similar manner as described in Example 6A above, Boc-D-Trp-O-pentyl (7.49 g, yield=61%) was prepared from the reaction of Boc-D-Trp-OH (10.0 g, 32.8 mmol), pentanol (2.90 g, 32.8 mmol) with HOBt hydrate (5.53 g, 36.1 mmol), and EDCI (6.93 g, 36.1 mmol) in dichloromethane (100 mL) and DMF (100 mL) at room temperature for overnight. ¹H NMR (CDCl₃, 400 MHz) δ (ppm): 8.07 (br. s, 1H), 7.57 (d, J=8.1 Hz, 1H), 7.35 (d, J=8.1 Hz, 1H), 7.19 (t, J=7.1 Hz, 1H), 7.08-7.15 (m, 1H), 7.01 (s, 1H), 5.08 (d, J=8.1 Hz, 1H), 4.57-4.70 (m, 1H), 3.95-4.14 (m, 2H), 3.20-3.38 (m, 2H), 1.50-1.61 (m, 2H), 1.15-1.47 (m, 13H), 0.87 (t, J=7.1 Hz, 3H).

### B. Preparation of H-D-Trp-O-pentyl hydrochloride

Proceeding in a similar manner as described in Example 6B above, H-D-Trp-O-2-pentyl hydrochloride (4.68 g, yield=75%) was prepared from the deprotection of Boc-D-Trp-O-pentyl (5.64 g, 13.6 mmol) with HCl gas in a solvent mixture of ether (75 mL) and ethyl acetate under ice-water bath cooling. ¹H NMR (DMSO-D₆, 400 MHz) δ (ppm): 11.12 (br. s, 1H), 8.64 (br. s, 3H), 7.52 (d, J=8.1 Hz, 1H), 7.37 (d, J=8.1 Hz, 1H), 7.25 (s, 1H), 7.06-7.17 (m, 1H), 6.93-7.06 (m, 1H), 4.19 (t, J=6.1 Hz, 1H), 3.86-4.10 (m, 2H), 3.15-3.38 (m, 2H), 1.32-1.52 (m, 2H), 1.14-1.28 (m, 2H), 1.01-1.13 (m, 2H), 0.82 (m, 3H); MS-ESI (m/z): 275 [M+1]⁺ (free base).

### C. Preparation of Boc-D-Glu(D-Trp-O-pentyl)-O-isoamyl

Proceeding in a similar manner as described in Example 6E above, Boc-D-Glu(D-Trp-O-pentyl)-O-isoamyl (1.44 g, yield=88%) was prepared from the reaction of H-D-Trp-O-pentyl hydrochloride (932 mg, 3.0 mmol), EDCI (933 mg, 3.3 mmol), HOBt hydrate (505 mg, 7.9 mmol), DIPEA (426 mg, 3.3 mmol) and Boc-D-Glu(OH)—O-isoamyl (952 mg, 3.0 mmol) in DMF (10 mL) at room temperature. ¹H NMR (CDCl₃, 400 MHz) δ (ppm): 8.15 (br. s, 1H), 7.53 (d, J=8.1 Hz, 1H), 7.35 (d, J=8.1 Hz, 1H), 7.18 (t, J=7.1 Hz, 1H), 7.06-7.15 (m, 1H), 7.02 (br. s, 1H), 6.24 (d, J=6.1 Hz, 1H), 5.23 (d, J=7.1 Hz, 1H), 4.85-4.98 (m, 1H), 3.93-4.28 (m, 5H), 3.21-3.42 (m, 2H), 2.10-2.32 (m, 3H), 1.82-1.98 (m, 1H), 1.62-1.74 (m, 1H), 1.47-1.62 (m, 4H), 1.43 (s, 9H), 1.15-1.37 (m, 4H), 0.82-0.97 (m, 9H); MS-ESI (m/z): 574 [M+1]⁺.

### D. Preparation of H-D-Glu(D-Trp-O-pentyl)-O-isoamyl hydrochloride

Proceeding In a similar manner as described under Example 6F above, H-D-Glu(D-Trp-O-pentyl)-O-isoamyl hydrochloride (900 mg, yield=58%) was obtained from the deprotection of Boc-D-Glu(D-Trp-O-pentyl)-O-isoamyl (1.41 g, 2.4 mmol) with a 2M HCl in ether solution (15 mL). ¹H NMR (DMSO-D₆, 400 MHz) δ (ppm): 10.99 (br. s, 1H), 8.72 (br. s, 3H), 8.55 (d, J=5.1 Hz, 1H), 7.47 (d, J=7.1 Hz, 1H), 7.34 (d, J=7.1 Hz, 1H), 7.19 (s, 1H), 7.04 (d, J=7.1 Hz, 1H), 6.92-7.01 (m, 1H), 4.40-4.54 (m, 1H), 4.08-4.23 (m, 2H), 3.83-4.02 (m, 3H), 2.98-3.22 (m, 2H), 2.21-2.45 (m, 2H), 1.91-2.09 (m, 2H), 1.58-1.73 (m, 1H), 1.35-1.54 (m, 4H), 1.05-1.29 (m, 4H), 0.75-0.93 (m, 9H); MS-ESI (m/z): 474 [M+1]⁺ (free base).

### Example 8

### Preparation of H-D-Glu(D-Trp-OEt)-O-isoamyl hydrochloride (Apo918.HCl)

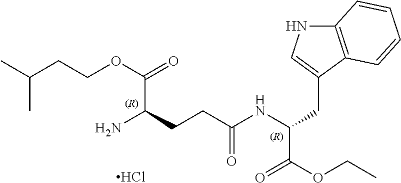

### A. Preparation of Boc-D-Glu(D-Trp-O-Et)-O-isoamyl

Proceeding in a similar manner as described in Example 6E above, Boc-D-Glu(D-Trp-O-Et)-O-isoamyl (870 mg, yield=54%) was prepared from the reaction of H-D-Trp-O-Et hydrochloride (806 mg, 3.0 mmol), EDCI (933 mg, 3.3 mmol), HOBt hydrate (505 mg, 7.9 mmol), DIPEA (426 mg, 3.3 mmol) and Boc-D-Glu-O-isoamyl (952 g, 3.0 mmol) in DMF (10 mL) at room temperature. ¹H NMR (CDCl₃, 400 MHz) δ (ppm): 8.18 (br. s, 1H), 7.53 (d, J=8.1 Hz, 1H), 7.35 (d, J=8.1 Hz, 1H), 7.18 (t, J=7.6 Hz, 1H), 7.06-7.15 (m, 1H), 7.02 (s, 1H), 6.24 (d, J=7.1 Hz, 1H), 5.24 (d, J=8.1 Hz, 1H), 4.81-5.00 (m, 1H), 4.00-4.29 (m, 5H), 3.22-3.43 (m, 2H), 2.06-2.34 (m, 3H), 1.81-1.97 (m, 1H), 1.57-1.76 (m, 1H), 1.48-1.56 (m, 2H), 1.43 (s, 9H), 1.22 (t, J=7.1 Hz, 3H), 0.91 (d, J=5.1 Hz, 6H); MS-ESI (m/z): 532 [M+1]⁺.

### B. Preparation of H-D-Glu(D-Trp-O-Et)-O-isoamyl hydrochloride

Proceeding In a similar manner as described under Example 6F, H-D-Glu(D-Trp-OEt)-O-isoamyl hydrochloride (Apo918.HCl, 240 mg, yield=55%) was obtained from the deprotection of Boc-D-Glu(D-Trp-O-Et)-O-isoamyl (515 mg, 1.0 mmol) with a 1M HCl in ether solution (12 mL). ¹H NMR (DMSO-D₆, 400 MHz) δ (ppm): 10.85 (br. s, 1H), 8.27 (d, J=7.1 Hz, 1H), 7.49 (d, J=8.1 Hz, 1H), 7.33 (d, J=7.1 Hz, 1H), 7.14 (s, 1H), 7.06 (t, J=7.6 Hz, 1H), 6.93-7.02 (m, 1H), 4.37-4.54 (m, 1H), 3.89-4.12 (m, 4H), 3.17-3.26 (m, 1H), 3.07-3.17 (m, 1H), 2.93-3.07 (m, 1H), 2.19 (t, J=7.1 Hz, 2H), 1.37-1.87 (m, 7H), 1.07 (t, J=7.1 Hz, 3H), 0.88 (d, J=7.1 Hz, 6H); MS-ESI (m/z): 432 [M+1]⁺ (free base).

### Example 9

### Preparation of H-D-Glu(D-Trp-O-isoamyl)-O-Et hydrochloride (Apo923.HCl)

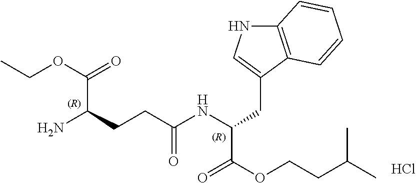

### A. Preparation of Boc-D-Trp-O-isoamyl

Proceeding in a similar manner as described under Example 6A, Boc-D-Trp-O-isoamyl was prepared as a white solid (18.58 g) from the reaction of Boc-D-Trp-OH (25.00 g, 82.2 mmol), 3-methylbutan-1-ol (7.97 g, 90.4 mmol), EDCI (18.90 g, 98.9 mmol), HOBt hydrate (12.58 g, 82.2 mmol) and Et₃N (18.29 g, 180.7 mmol) in DMF (250 mL). Yield=60%; ¹H NMR (DMSO-D₆, 400 MHz) δ (ppm): 10.86 (br. s, 1H), 7.48 (d, J=8.1 Hz, 1H), 7.34 (d, J=8.1 Hz, 1H), 7.22 (d, J=8.1 Hz, 1H), 7.16 (s, 1H), 7.03-7.11 (m, 1H), 6.94-7.03 (m, 1H), 4.13-4.24 (m, 1H), 3.92-4.08 (m, 2H), 2.90-3.16 (m, 2H), 1.44-1.62 (m, 1H), 1.34 (s, 10H), 1.24 (br. s, 1H), 0.82 (t, J=6.6 Hz, 6H); MS-ESI (m/z): 375 [M+1]⁺.

### B. Preparation of H-D-Trp-O-isoamyl hydrochloride

Proceeding in a similar manner as described under Example 6B, H-D-Trp-O-isoamyl hydrochloride (12.0 g) was obtained as an off-white solid after bubbling HCl gas for 2 h into a mixture of Boc-D-Trp-O-isoamyl (18.00 g, 48.1 mmol) in ethyl acetate (100 mL) and ether (100 mL) under ice-water bath cooling. Yield=80%. ¹H NMR (DMSO-D₆, 400 MHz) δ (ppm): 11.09 (br. s, 1H), 8.47 (br. s, 3H), 7.50 (d, J=7.1 Hz, 1H), 7.38 (d, J=8.1 Hz, 1H), 7.23 (s, 1H), 7.10 (t, J=7.1 Hz, 1H), 6.96-7.06 (m, 1H), 4.23 (t, J=6.6 Hz, 1H), 3.95-4.13 (m, 2H), 3.18-3.31 (m, 2H), 1.36-1.52 (m, 1H), 1.24-1.36 (m, 2H), 0.79 (d, J=5.1 Hz, 6H); MS-ESI (m/z): 275 [M+1]⁺ (free base).

### C. Preparation of Boc-D-Glu(D-Trp-O-isoamyl)-O-Et

Proceeding in a similar manner as described under Example 6E, Boc-D-Glu(D-Trp-O-isoamyl)-O-Et was prepared from the reaction of Boc-D-Glu(OH)—O-ethyl dicyclohexylamine (2.94 g, 6.4 mmol), H-D-Trp-O-isoamyl hydrochloride (2.00 g, 6.4 mmol), EDCI (1.48 g, 7.7 mmol), HOBt hydrate (0.99 g, 6.4 mmol) and Et₃N (2.28 g, 22.5 mmol) in DMF (25 mL). Yield=58%; ¹H NMR (DMSO-D₆, 400 MHz) δ (ppm): 10.86 (br. s, 1H), 8.29 (d, J=7.1 Hz, 1H), 7.49 (d, J=8.1 Hz, 1H), 7.35 (d, J=8.1 Hz, 1H), 7.24 (d, J=7.1 Hz, 1H), 7.15 (s, 1H), 7.08 (t, J=7.6 Hz, 1H), 6.99 (t, J=7.6 Hz, 1H), 4.44-4.54 (m, 1H), 4.02-4.15 (m, 3H), 3.98 (t, J=6.6 Hz, 2H), 2.98-3.19 (m, 2H), 2.20 (br. s, 2H), 1.90 (d, J=6.1 Hz, 1H), 1.64-1.81 (m, 1H), 1.43-1.52 (m, 1H), 1.39 (s, 8H), 1.33 (br. s, 3H), 1.18 (t, J=7.1 Hz, 3H), 0.81 (t, J=6.6 Hz, 6H); MS-ESI (m/z): 532 [M+1]⁺.

### D. Preparation of H-D-Glu(D-Trp-O-isoamyl)-O-Et hydrochloride (Apo923.HCl)

Proceeding in a similar manner as described under Example 6F, H-D-Glu(D-Trp-O-isoamyl)-O-Et hydrochloride (Apo923.HCl) was obtained as an off-white foam (250 mg) from the deprotection of Boc-D-Glu(D-Trp-O-isoamyl)-O-Et (0.60 g, 1.1 mmol) with a 2M HCl in ether solution (10 mL). Yield=47%; ¹H NMR (DMSO-D₆, 400 MHz) δ (ppm): 10.94 (br. s, 1H), 8.59 (br. s, 3H), 8.51 (d, J=7.1 Hz, 1H), 7.48 (d, J=7.1 Hz, 1H), 7.35 (d, J=8.1 Hz, 1H), 7.18 (s, 1H), 7.07 (t, J=7.6 Hz, 1H), 6.99 (t, J=7.1 Hz, 1H), 4.48 (q, J=7.1 Hz, 1H), 4.17 (d, J=5.1 Hz, 2H), 3.89-4.03 (m, 3H), 2.98-3.18 (m, 2H), 2.21-2.42 (m, 2H), 1.93-2.03 (m, 2H), 1.41-1.54 (m, 1H), 1.28-1.36 (m, 2H), 1.21 (t, J=7.1 Hz, 3H), 0.81 (t, J=6.6 Hz, 6H); MS-ESI (m/z): 432 [M+1]+(free base).

### Example 10

### Preparation of H-D-Glu(D-Trp-O-isoamyl)-O-Bzl hydrochloride (Apo924.HCl)

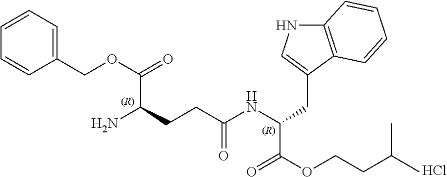

### A. Preparation of Boc-D-Glu(D-Trp-O-isoamyl)-O-Bzl

Proceeding in a similar manner as described in Example 6E above, Boc-D-Glu(D-Trp-O-isoamyl)-O-Bzl (3.2 g, yield=83%) was prepared from the reaction of H-D-Trp-O-isoamyl hydrochloride (2.00 g, 3.0 mmol), EDCI (1.48 g, 7.7 mmol), HOBt hydrate (0.99 g, 6.4 mmol), Et₃N (2.28 g, 22.5 mmol) and Boc-D-Glu(OH)—O-Bzl (2.17 g, 6.4 mmol) in DMF (25 mL) at room temperature. ¹H NMR (DMSO-D₆, 400 MHz) δ (ppm): 10.86 (br. s, 1H), 8.29 (d, J=7.1 Hz, 1H), 7.47 (d, J=8.1 Hz, 1H), 7.29-7.39 (m, 7H), 7.13 (s, 1H), 7.06 (t, J=7.6 Hz, 1H), 6.98 (t, J=7.1 Hz, 1H), 5.05-5.19 (m, 2H), 4.41-4.51 (m, 1H), 4.03 (q, J=7.1 Hz, 2H), 2.96-3.15 (m, 2H), 2.11-2.29 (m, 2H), 1.84-1.97 (m, 1H), 1.74 (d, J=7.1 Hz, 1H), 1.40-1.50 (m, 1H), 1.38 (br. s, 8H), 1.22-1.34 (m, 4H), 0.78 (t, J=6.6 Hz, 6H); MS-ESI (m/z): 594 [M+1]⁺.

### B. Preparation of H-D-Glu(D-Trp-O-isoamyl)-O-Bzl hydrochloride (Apo924.HCl)

Proceeding In a similar manner as described under Example 6F above, H-D-Glu(D-Trp-O— isoamyl)-O-Bzl hydrochloride (0.59 g, yield=55%) was obtained from the deprotection of Boc-D-Glu(D-Trp-O-isoamyl)-O-Bzl (1.2 g, 2.0 mmol) with a 2M HCl in ether solution (18 mL). ¹H NMR (DMSO-D₆, 400 MHz) δ (ppm): 10.92 (s, 1H), 8.57 (br. s, 3H), 8.49 (d, J=7.1 Hz, 1H), 7.47 (d, J=8.1 Hz, 1H), 7.32-7.42 (m, 6H), 7.16 (s, 1H), 7.07 (t, J=7.1 Hz, 1H), 6.98 (t, J=7.1 Hz, 1H), 5.12-5.31 (m, 2H), 4.48 (q, J=7.1 Hz, 1H), 4.07 (d, J=5.1 Hz, 1H), 3.91-4.01 (m, 2H), 2.99-3.19 (m, 2H), 2.24-2.43 (m, 2H), 1.89-2.08 (m, 2H), 1.38-1.52 (m, 1H), 1.24-1.36 (m, 2H), 0.74-0.84 (m, 6H); MS-ESI (m/z): 494 [M+1]+(free base).

### Example 11

### Preparation of gamma-D-glutamyl-L-tryptophan diethyl ester hydrochloride or ethyl (2R)-2-amino-5-{[(2S)-1-(ethoxy)-3-(1H-indol-3-yl)-1-oxopropan-2-yl]amino}-5-oxopentanoate hydrochloride or H-D-Glu(L-Trp-O-ethyl)-O-ethyl.HCl. or Apo870 hydrochloride

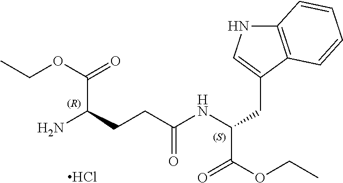

In a similar manner as described in Example 2, H-D-Glu(L-Trp-OH)—OH was reacted with HCl in ethanol to give gamma-D-glutamyl-L-tryptophan diethyl ester hydrochloride. The HPLC method described in Example 2 was used. HPLC RT=11.3 min; HPLC (AUC) purity at 280 nm=96.8%; ¹H NMR (DMSO-d₆, 400 MHz) δ ppm: 10.91 (s, 1H), 8.51 (d, J=7.3 Hz, 1H), 7.80-8.40 (br, m 3H), 7.49 (d, J=7.8 Hz, 1H), 7.34 (d, J=8.0 Hz, 1H), 7.17 (s, 1H), 7.07 (t, J=7.4 Hz, 1H), 6.99 (t, J=7.4 Hz, 1H), 4.44-4.47 (m, 1H), 4.16-4.21 (q, J=7.0 Hz, 2H), 3.99-4.05 (q, J=7.0 Hz, 2H), 3.91-3.95 (m, 1H), 3.01-3.16 (m, 2H), 2.33-2.39 (m, 1H), 2.21-2.25 (m, 1H), 1.90-1.98 (m, 2H), 1.22 (t, J=7.0 Hz, 3H), 1.08 (t, J=7.0 Hz, 3H); MS-ESI(m/z) 390 [M+1]⁺ (free base).

### Example 12

### Preparation of (R)-ethyl 5-((S)-3-(1H-indol-3-yl)-1-(isopentyloxy)-1-oxopropan-2-ylamino)-2-amino-5-oxopentanoate hydrochloride or H-D-Glu(L-Trp-O-isoamyl)-O-ethyl hydrochloride (Apo914.HCl)

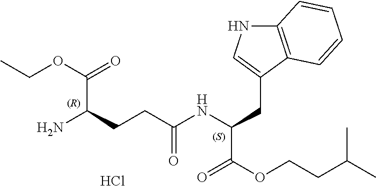

### A. Preparation of Boc-L-Trp-O-isoamyl

Boc-D-Trp-OH (10.0 g, 32.8 mmol), 3-methyl-1-butanol (7.1 mL, 65.7 mmol), EDCI (8.2 g, 42.7 mmol), HOBt (5.3 g, 39.4 mmol) and DIPEA (7.4 mL, 42.7 mmol) were mixed in and DMF (100 mL). The resulting mixture was stirred at room temperature for overnight. The reaction mixture was poured into a beaker of cold water (100 mL) with stirring, and the resulting suspension was stirred at 5° C. (ice bath) for 20 min. Suction filtration afforded Boc-L-Trp-O-isoamyl as a white solid, which was air-dried for overnight (10.8 g). Yield=88%; ¹H NMR (DMSO-d₆, 400 MHz) δ ppm: 10.86 (br. s., 1H), 7.48 (d, J=8.1 Hz, 1H), 7.34 (d, J=8.1 Hz, 1H), 7.22 (d, J=7.1 Hz, 1H), 7.16 (s, 1H), 7.07 (t, J=7.1 Hz, 1H), 6.99 (t, J=7.6 Hz, 1H), 4.12-4.24 (m, 1H), 3.93-4.09 (m, 2H), 3.05-3.15 (m, 1H), 2.95-3.05 (m, 1H), 1.48-1.59 (m, 1H), 1.31-1.41 (m, 11H), 0.82 (t, J=6.6 Hz, 6H); MS-ESI (m/z) 375 [M+1]⁺.

### B. Preparation of H-L-Trp-O-isoamyl hydrochloride

HCl gas was bubbled into a suspension of Boc-L-Trp-O-isoamyl (10.52 g, 28.1 mmol) in 150 ml EtOAc for 1.5 h. The suspension was stirred at 5° C. (ice-bath) for 20 min. The solid product was collected by suction filtration, and washed with EtOAc (3×15 mL) to afford H-L-Trp-O-isoamyl hydrochloride as white solid (7.83 g). Yield: 90%; ¹H NMR (DMSO-d₆, 400 MHz) δ ppm: 11.13 (br. s., 1H), 8.66 (br. s., 2H), 7.52 (d, J=8.1 Hz, 1H), 7.38 (d, J=8.1 Hz, 1H), 7.25 (s, 1H), 7.09 (t, J=7.6 Hz, 1H), 7.01 (t, J=7.6 Hz, 1H), 4.19 (t, J=6.6 Hz, 1H), 3.94-4.08 (m, 2H), 3.33 (d, J=5.1 Hz, 1H), 3.20-3.29 (m, 1H), 1.36-1.48 (m, 1H), 1.23-1.33 (m, 2H), 0.78 (d, J=5.1 Hz, 6H); MS-ESI (m/z) 275 [M+1]⁺ (free base).

### C. Preparation of Boc-D-Glu(L-Trp-O-isoamyl)-O-Bzl

To a solution of Boc-D-Glu-O-Bzl (8.3 g, 24.6 mmol), H-L-Trp-O-isoamyl hydrochloride (7.65 g, 24.6 mmol), EDCI (5.67 g, 29.5 mmol.), and HOBt (3.5 g, 25.8 mmol) in DMF (100 mL) under ice-water bath cooling, was added DIPEA (8.6 mL, 49.2 mmol). The resulting mixture was stirred at room temperature for overnight. The reaction mixture was poured into a beaker of cold water (250 mL) with stirring. The mixture was extracted with ethyl acetate (100 mL×3). The combined organic layers was successively washed with a 10% citric acid solution (30 mL), a saturated NaHCO₃ (50 mL) and brine (50 mL), and was then dried over MgSO₄. After solvent was removed in vacuo, Boc-D-Glu(L-Trp-O-isoamyl)-O-bzl was obtained as light yellowish oil (13.5 g). Yield=93%; ¹H NMR (DMSO-d₆, 400 MHz) δ ppm: 10.87 (br. s., 1H), 8.30 (d, J=7.1 Hz, 1H), 7.48 (d, J=8.1 Hz, 1H), 7.27-7.40 (m, 7H), 7.15 (br. s., 1H), 7.07 (t, J=7.6 Hz, 1H), 6.91-7.03 (m, 1H), 5.04-5.19 (m, 2H), 4.48 (d, J=6.1 Hz, 1H), 3.97 (t, J=6.1 Hz, 3H), 3.12 (dd, J=14.1, 6.1 Hz, 1H), 3.02 (dd, J=14.1, 8.1 Hz, 1H), 2.14-2.29 (m, 2H), 1.93 (d, J=8.1 Hz, 1H), 1.67-1.83 (m, 1H), 1.41-1.55 (m, 2H), 1.28-1.38 (m, 10H), 0.80 (t, J=6.1 Hz, 6H); MS-ESI (m/z) 594 [M+1]⁺.

### D. Preparation of Boc-D-Glu(L-Trp-O-isoamyl)-OH

A mixture of Boc-D-Glu(L-Trp-O-isoamyl)-O-benzyl (12.35 g, 20.8 mmol) and 1.5 g of 10% Pd on activated carbon (wet) in ethanol (250 ml) was shaken in a Parr apparatus under a hydrogen atmosphere at a pressure of 45 psi at room temperature for 2 h. The Pd catalyst was filtered through Celite™ and the filtrate was evaporated under reduced pressure to give a pink oil, which was dried under vacuum to afford Boc-D-Glu(L-Trp-O-isoamyl)-OH (9.1 g) as a pink foamy solid. Yield=87%; ¹H NMR (DMSO-d₆, 400 MHz) 8 ppm: 10.87 (s, 1H), 8.30 (d, J=7.1 Hz, 1H), 7.48 (d, J=7.1 Hz, 1H), 7.34 (d, J=8.1 Hz, 1H), 7.15 (s, 1H), 7.03-7.12 (m, 2H), 6.93-7.03 (m, 1H), 4.41-4.54 (m, 1H), 3.98 (t, J=6.6 Hz, 2H), 3.82-3.92 (m, 1H), 3.39-3.50 (m, 2H), 3.07-3.18 (m, 1H), 2.97-3.07 (m, 1H), 2.18 (t, J=7.6 Hz, 2H), 1.90 (d, J=8.1 Hz, 1H), 1.70 (dd, J=13.6, 7.6 Hz, 1H), 1.47 (dq, J=13.3, 6.7 Hz, 1H), 1.26-1.41 (m, 9H), 1.07 (t, J=6.6 Hz, 1H), 0.75-0.84 (m, 6H); MS-ESI (m/z) 504 [M+1]⁺.

### E. Preparation of Boc-D-Glu(L-Trp-O-isoamyl)-O-ethyl

To a solution of Boc-D-Glu(L-Trp-O-isoamyl)-OH (1.25 g, 2.48 mmol) in DMF (35 mL) was successively added iodoethane (0.6 mL, 7.45 mmol) and potassium carbonate (0.69 g, 4.96 mmol) at room temperature. The resulting mixture was stirred at room temperature for overnight. The reaction mixture was quenched with water (25 mL), and then extracted with EtOAc (50 mL×3). The combined organic layers was successively washed with a 10% citric acid solution (20 mL), a saturated NaHCO₃ solution and brine (25 mL), and the organic phase was dried over Na₂SO₄. After solvent was removed in vacuo, Boc-D-Glu(L-Trp-O-isoamyl)-O-ethyl (1.12 g) was obtained as a pinkish brown oil. Yield: 85%; ¹H NMR (DMSO-d₆, 400 MHz) δ ppm: 10.86 (s, 1H), 8.29 (d, J=7.1 Hz, 1H), 7.96 (s, 1H), 7.48 (d, J=7.1 Hz, 1H), 7.34 (d, J=8.1 Hz, 1H), 7.22 (d, J=8.1 Hz, 1H), 7.14 (s, 1H), 7.07 (t, J=7.6 Hz, 1H), 6.99 (t, J=7.6 Hz, 1H), 4.47 (d, J=7.1 Hz, 1H), 4.03-4.16 (m, 2H), 3.98 (t, J=7.1 Hz, 2H), 3.91 (d, J=5.1 Hz, 1H), 3.07-3.16 (m, 1H), 3.04 (d, J=9.1 Hz, 1H), 2.18 (t, J=7.6 Hz, 2H), 1.79-1.97 (m, 1H), 1.63-1.78 (m, 1H), 1.43-1.54 (m, 1H), 1.27-1.38 (m, 10H), 1.18 (t, J=7.1 Hz, 3H), 0.81 (t, J=6.6 Hz, 6H); MS-ESI (m/z) 532[M+1]⁺.

### F. Preparation of H-D-Glu(L-Trp-O-isoamyl)-O-ethyl hydrochloride (Apo914.HCl)

HCl gas was bubbled into a solution of Boc-D-Glu(L-Trp-O-isoamyl)-O-ethyl (1.05 g, 1.98 mmol) in 35 mL of dichloromethane for 2 h. The reaction mixture was evaporated to dryness and the crude product was purified by flash chromatography on silica gel using a solvent mixture of isopropanol and dichloromethane (1/1 ratio, v/v) as eluent. The resulting sticky foamy solid was dissolved in a 2M HCl in Et₂O solution, and stirred at room temperature for 30 min. After removal of volatile materials by evaporation under reduced pressure, H-D-Glu(L-Trp-O-isoamyl)-O-ethyl hydrochloride (Apo914.HCl) was obtained as a brown -pinkish foamy solid (0.81 g). Yield=88%; ¹H NMR (DMSO-d₆, 400 MHz) δ ppm: 10.90 (br. s., 1H), 8.43 (d, J=7.07 Hz, 1H), 7.48 (d, J=8.08 Hz, 1H), 7.34 (d, J=8.08 Hz, 1H), 7.16 (s, 1H), 7.03-7.11 (m, 1H), 6.94-7.02 (m, 1H), 4.47 (q, J=7.07 Hz, 1H), 4.13 (d, J=7.07 Hz, 2H), 4.08-4.20 (m, 2H), 3.94-4.03 (m, 2H), 3.57-3.68 (m, 1H), 3.13 (dd, J=6.06, 14.15 Hz, 1H), 3.03 (dd, J=8.59, 14.65 Hz, 1H), 2.12-2.37 (m, 2H), 1.82-1.95 (m, 1H), 1.68-1.82 (m, 1H), 1.48 (dt, J=6.57, 13.14 Hz, 1H), 1.26-1.38 (m, 2H), 1.21 (t, J=7.07 Hz, 3H), 0.75-0.86 (m, 6H); MS-ESI (m/z) 432[M+1]⁺ (free base).

### Example 13

### Preparation of Preparation of H-D-Glu(L-Trp-O-isoamyl)-O-Bzl hydrochloride (Apo927.HCl)

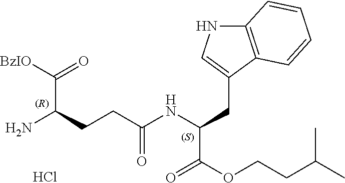

Boc-D-Glu(L-Trp-O-isoamyl)-O-bzl (prepared as described in Example 12C) (0.97 g, 1.63 mmol) was stirred in 10 mL of 4 M HCl in dioxane at room temperature for 30 min. The reaction mixture was evaporated to dryness and the residual oil was taken up in acetonitrile. The mixture was again evaporated to dryness, and the residual foamy solid was dried under vacuum for 4 h. Thus, H-D-Glu(L-Trp-O-isoamyl)-O-Bzl hydrochloride (0.80 g) was obtained in 92% yield. ¹H NMR (CDCl₃, 400 MHz) δ ppm: 9.12 (br. s., 1H), 8.03 (s, 1H), 7.47 (d, J=7.1 Hz, 1H), 7.27-7.34 (m, 2H), 7.24 (br. s., 3H), 7.19 (br. s., 2H), 6.98-7.12 (m, 2H), 4.90-5.06 (m, 2H), 4.80 (d, J=4.0 Hz, 1H), 3.97-4.09 (m, 3H), 3.75-3.82 (m, 1H), 3.62-3.70 (m, 1H), 3.22-3.31 (m, 1H), 3.11-3.21 (m, 1H), 2.46 (br. s., 1H), 2.33-2.42 (m, 1H), 2.26 (br. s., 1H), 2.18 (br. s., 1H), 1.60 (dt, J=13.1, 6.6 Hz, 1H), 1.40-1.50 (m, 2H), 0.87 (d, J=6.1 Hz, 6H); MS-ESI (m/z) 494[M+1]+(free base).

### Example 14

### Distribution Coefficient Determination, D_{7.4}

MOPS buffer (50 mM, pH=7.4) and 1-octanol were used as the aqueous phase and the organic phase, respectively. The MOPS buffer and 1-octanol were mixed, and pre-saturated with each other prior to use.

In a typical experiment, an aqueous solution of Apo848 hydrochloride salt (H-D-Glu(D-Trp-O-isoamyl)-O-isoamy HCl) was prepared by weighing out 2 mg of the compound into a 5-mL volumetric flask, followed by addition of MOPS buffer (50 mM, pH=7.4) to volume. The resulting mixture was sonicated and vortexed to ensure complete dissolution. The resulting solution was analyzed by HPLC (Column: XTerra MS C₁₈, 5 μM, 4.6×250 mm; Mobile phase: A=4 mM Tris, 2 mM EDTA, pH 7.4 aqueous, B=acetonitrile; Gradient method: time in minutes—B in %:0-5, 15-55, 25-55, 25.05-5, 30-5; Flow rate: 1 mL/min; Injection volume=2 μL; detector wavelength: 282 nm) to obtain the peak height (H_{aqu}^{l}).

One mL of this aqueous solution was pipetted out into another 10-mL test-tube and mixed with 1 mL of 1-octanol. The mixture was vortexed for 1 hour, then centrifuged at 4000 rpm for 15 minutes. The two phases were separated. Both the aqueous phase and the organic phase were analyzed by HPLC to obtain the peak heights, H_{aqu}^{F} and H_{org}^{F}. The distribution coefficient, D_{7.4}, was calculated using one or both the following equations: D_{7.4}=(H_{aqu}^{l}−H_{aqu}^{F})/H_{aqu}^{F}, or D_{7.4}=H_{org}^{F}/H_{aqu}^{F}.

The D_{7.4} of Apo848 was determined to be 127, and hence the logD_{7.4} was calculated to be 2.1. In a similar fashion, the log D_{7.4} of the following compounds H-D-Glu(D-Trp-O-Me)-O-Me (0.57), H-D-Glu(D-Trp-O-Me)-OH (−0.89) and H-D-Glu(D-Trp-OH)—OH (−3.22) were determined.

### Example 15

### Biotransformation Studies of a Compound of Formula I in Human Hepatocytes General Procedure

LiverPool® cryopreserved human hepatocytes (pooled from 10 male donors) was obtained from Celsis In Vitro Technologies. The hepatocytes were stored in liquid nitrogen until used. Just before the assay, the hepatocytes were quickly thawed at 37° C. and centrifuged at 100×g for 10 min. The media was removed and cells were re-suspended in PBS at a density of 4×10⁶ cells/mL.

The compound of Formula I (100 μM) was incubated with 0.1×10⁶ hepatocytes in 50 μL volume. After 10, 20, 60, 120 and 240 min of incubation, the reaction was quenched by adding an equal volume of 5% (w/v) TCA. The “time 0” sample was generated by adding TCA before the test compound. After brief vortexing and 10-min incubation on ice, samples were centrifuged (16,000×g, 10 min) and the supernatants were analyzed by HPLC with UV detection.

HPLC analysis of pro-drugs in SGF, SIF, plasma and hepatocytes samples: HPLC analysis was done using an Agilent 1100 series HPLC system consisting of a programmable multi-channel pump, auto-injector, vacuum degasser and HP detector controlled by Agilent HPLC218 Chem Station Rev.A.09.03 software for data acquisition and analysis. A gradient method was used for the determination of all pro-drugs and their hydrolysis products including Apo805 on an Agilent Eclipse XDB, C18 column (part #963967-902, 150×4.6 mm, 3.5 μm) with the following chromatographic conditions:
Temperature: Ambient
Mobile phase: A=Aqueous phase: 10 mM Tris-HCl, 2 mM EDTA, pH 7.4
   B=Organic phase: Acetonitrile
Gradient method: Time: 0 min 5% B, 25 min 50% B, 35 min 80% B, 45 min 5% B, 50 min 5% B.
Mobile phase flow rate: 1.0 mL/min
Injection volume: 50 μL
Data acquisition time: 30 min
Detection wavelength: 280 nm; 4 nm bandwidth, ref. 360 nm, 4 nm bandwidth

The chromatograms at λ=280 nm were analyzed. Peak area (mAU*s) was used for quantitation of pro-drugs, intermediates and H-D-Glu(D-Trp-OH)—OH (Apo805).

When the bioconversion of Apo848, a compound of Formula IA wherein G=T=isoamyl, was studied in vitro by incubation with human cryopreserved hepatocytes, HPLC analysis of the incubation mixture confirmed the formation of Apo805 in 45% after 3 h. Apo848 shows significant improvement over another compound Apo804 (H-D-Glu(D-Trp-OMe)-O—CH₂Ph which has a 30% conversion to Apo805 in the same hepatocyte system after 3 h.

### Example 16

### Pharmacokinetic Studies of a Compound of Formula I in Rats

### General Procedure for Animal Dosing

Groups of five male Sprague-Dawley rats weighing 250 to 300 g were utilized per dosing group. One day prior to dosing, venous and arterial catheters (made of 20 cm long polyurethane coiled tubing, and filled with 100 units/mL heparinized saline) were implanted into the jugular vein and carotid artery of each rat. Rats were fasted overnight prior to oral dosing and fed approximately 2 hours post-dosing. All dosing and blood sampling was performed on fully conscious rats. Tested compounds were administered either by oral gavage as solutions in water, or by intravenous injection (Apo805K1 only) as solution in 0.9% sodium chloride, final pH 7.0, at doses equivalent to 5 mg/kg (per Apo805 content). Blood (0.3 mL) was sampled from each animal from the carotid artery for up to 30 hours post-dosing, each sampling followed by an equivalent naive-blood replacement. The blood sample was immediately centrifuged (4300×g for 5 minutes at 4° C.), and frozen at −80° C. until LC/MS/MS analysis.

### General Procedure for LC-MS/MS Analysis of Plasma Drug Concentration

Metanol (200 μL) was added to plasma samples (50 μL) to precipitate plasma proteins. After brief vortexing and centrifugation, the supernatant (200 uL) was removed and dried at 40° C. under a stream if nitrogen. The sample was reconstituted in water (300 μL) and 25 μL was injected for analysis.

A Sciex API 365 LC/MS/MS spectrophotometer equipped with Ionics EP10+ and HSID, was used. A chiral column (Supelco-Astec CHIROBIOTIC™ TAG), 100×2.1 mm, 5 μm was used at ambient temperature. The mobile phase consisted of 0.1% formic acid in water (A) and 0.1% formic acid in acetonitrile (B) in a ratio of 88:12(A:B; v/v) and the flow rate was 0.6 mL/min. Positive ion electrospray ionization (ESI+) in MRM mode was used for analysis. Samples were analysed for the concentration of Apo805.

### Oral Bioavailability of Apo848 and Apo805 (H-D-Glu(D-Trp-OH)—OH) in Rats

Absolute oral bioavailability of pro-drugs Apo848, a compound of Formula IA wherein G=T=isoamyl) was compared to that of Apo805K1 (potassium salt of H-D-Glu(D-Trp-OH)—OH) in male Sprague-Dawley rats. Adult animals, five per group, were dosed orally with 5 mg/kg Apo805K1, Apo848, or Apo838 and intravenously with 5 mg/kg Apo805K1. As Apo848 is instantaneously converted to Apo805 in rat blood, only levels of Apo805 were measured in plasma collected at various time intervals post-dosing.

### PK Analysis

Non-compartmental analysis was performed using WinNonlin 5.2 software, on individual animal data. Bioavailability was calculated as a ratio of AUC_{INF}_{_{—}}D after oral dosing of test compound to AUC_{INF}_{_{—}}D after IV dosing of Apo805K1.

FIG. 4 shows the plasma concentration of Apo805 after oral dosing of Apo848 or Apo805K1. Absolute oral bioavailability, calculated as a ratio of the area under the time-plasma concentration curve (AUC) after oral dosing to AUC after intravenous dosing was 48% for Apo848. Absolute bioavailability of Apo805K1 was only 12%. Thus, the bioavailability of pro-drugs is significantly enhanced compared to Apo805K1.

### Example 17

### Caco-2 Cell Permeability Evaluation of a Compound of Formula I

Human intestinal absorbtion potential of a compound of Formula I was estimated in caco-2 cells permeability assay.

### Cell Culture

Caco-2 cells obtained originally from ATCC were seeded onto 0.9-cm² PET filter (Becton Dickinson) at a density of 90000 cells/insert. Culture conditions were maintained for 21-28 days in 20% fetal bovine serum containing eagle's minimum essential medium enriched with non-essential amino acids. Integrity of the cell monolayers was evaluated via measurement of Lucifer Yellow paracellular apparent permeability coefficient (Papp).

### Transport Experiments

Prior to the addition of a test compound, growth medium was removed and monolayer was rinsed twice with Hank's balanced salt solution (HBSS) at 37° C. The filter inserts containing the cell monolayers were transferred to a separate 12-well cell culture plate containing HBSS or solution of the test compound in the bottom chamber. All drug transport experiments were performed at 37° C. using 50 μM solution of the test compound in HBSS at pH 7.4. The top chamber medium volume was 1 mL and the bottom chamber medium volume was 2 mL. For every experiment, the test compound solution was added to the top (apical-to-basolateral transport, A>B) or bottom (basolateral-to-apical transport, B>A) chamber and its appearance in the opposite chamber over time was monitored. A 100 μL sample was taken from the donor chamber immediately after the addition of the compound to confirm the initial concentration of the test compound (C₀). At 30, 60, 90 and 120 min, 100 μL of supernatant sample was removed from the receiving chamber followed by the addition of 100 μL of pre-heated buffer as replenishment. At 120 min, a 100 μL supernatant sample was taken from the donor chamber to determine the concentration of compound remaining at the end of experiment. Samples were analyzed by LC-MS/MS. In case of prodrugs which undergo partial hydrolysis during the experiment, the samples were analyzed for the concentration of the prodrug and all hydrolysis products.

### Permeability Calculations

The accumulated amount of a test compound appearing in the receiving chamber over time, dQ/dt, was used to calculate the apparent permeability (Papp) using the following equation: Papp=dQ/dt×1(A×C₀), where A is the area of the filter (0.9 cm²) and C₀ is the initial concentration of the test compound in the donor chamber. For test compounds that undergo partial hydrolysis during the experiment, the total amount (in moles) of transported material was used for calculations. For each test compound, Papp values for both A>B and B>A directions were therefore calculated using the slope of the steady-state rate constant dQ/dt for the respective direction. A high absorption potential was estimated from the Papp (A>B) if the value equaled to or was higher than 1.0×10⁶ cm/s. An efflux profile was indicated if the ratio Papp (B>A)/ Papp (A>B) equaled to or was higher than 2.5.

### Results

Human intestinal absorption potential of Apo848, a compound of Formula IA wherein G and T are isoamyl, was estimated in caco-2 permeability assay. The apparent permeability was 2.87×10⁻⁶ cm/s for Apo848, indicating a high permeability potential.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. Furthermore, numeric ranges are provided so that the range of values is recited in addition to the individual values within the recited range being specifically recited in the absence of the range. The word “comprising” is used herein as an open-ended term, substantially equivalent to the phrase “including, but not limited to”, and the word “comprises” has a corresponding meaning. As used herein, the singular forms “a”, “an” and “the” include plural references unless the context clearly dictates otherwise. Thus, for example, reference to “a thing” includes more than one such thing. Citation of references herein is not an admission that such references are prior art to the present invention. Furthermore, material appearing in the background section of the specification is not an admission that such material is prior art to the invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1. A compound of Formula I: 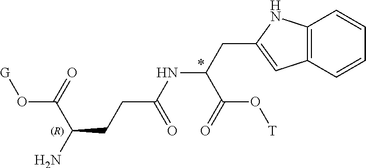
or a pharmaceutically acceptable salt thereof, wherein
G is selected from the group consisting of: C-i-Cs alkyl and benzyl;
T is selected from the group consisting of: Ci-Cs alkyl and benzyl; and * is a chiral carbon that is either in an (R) configuration or an (S) configuration,
provided that when * is in the (R) configuration, at least one of G and T is C5-C8 alkyl.

2. The compound of claim 1 wherein G is selected from the group consisting of: C₅-C₈ alkyl.

3. The compound of claim 1 wherein T is selected from C5-C8 alkyl.

4. The compound of claim 1 wherein * is in the (R) configuration.

5. The compound of claim 1 wherein * is in the (S) configuration.

6. The compound of claim 1 wherein G is isoamyl, T is isoamyl and * is in the (R) configuration.

7. The compound of claim 1 wherein G is isoamyl, T is isoamyl and * is in the (S) configuration.

8. The compound of claim 1 wherein G is heptyl, T is heptyl and * is in the (S) configuration.

9. The compound of claim 1 wherein G is pentyl, T is pentyl and * is in the (S) configuration.

10. The compound of claim 1 wherein G is hexyl, T is hexyl and * is in the (S) configuration.

11. The compound of claim 1 wherein G is isoamyl, T is pentyl and * is in the (R) configuration.

12. The compound of claim 1 wherein G is isoamyl, T is heptyl and * is in the (R) configuration.

13. The compound of claim 1 wherein G is isoamyl, T is ethyl and * is in the (R) configuration.

14. The compound of claim 1 wherein G is ethyl, T is ethyl and * is in the (S) configuration.

15. The compound of claim 1 wherein G is ethyl, T is isoamyl and * is in the (S) configuration.

16. The compound of claim 1 wherein G is ethyl, T is isoamyl and * is in the (R) configuration.

17. The compound of claim 1 wherein G is benzyl, T is isoamyl and * is in the (R) configuration.

18. The compound of claim 1 wherein G is benzyl, T is isoamyl and * is in the (S) configuration.

19. A pharmaceutical composition comprising the compound of claim 1 and a pharmaceutically acceptable excipient.
